(19) United States
(12) Patent Application Publication
(10) Pub. No.: US 2023/0069580 A1
(43) Pub. Date: Mar. 2, 2023

(54) CHEMICALLY CROSS-LINKED HYDROGEL AND ITS MICROSPHERES, PREPARATION METHOD AND APPLICATION

(71) Applicant: Zhongxin Biotechnology (Zhejiang) Co., Ltd., Zhejiang (CN)

(72) Inventors: Zhaozhu ZHENG, Zhejiang (CN); Xiaoqin WANG, Zhejiang (CN); Wenjun GUO, Zhejiang (CN)

(21) Appl. No.: 17/819,408

(22) Filed: Aug. 12, 2022

(30) Foreign Application Priority Data
Jun. 15, 2021 (CN) 202110660594.9

(51) Int. Cl. A61K 47/42; A61K 9/06; A61L 27/52; A61L 27/22

(52) U.S. Cl. CPC A61K 47/42; A61K 9/06; A61L 27/52; A61L 27/227; A61L 2430/02; A61L 2430/34; A61L 2300/622

(57) ABSTRACT

The chemically cross-linked hydrogel is a hydrogel formed by reaction of silk with a crosslinking agent, and the crosslinking agent is a diglycidyl ether crosslinking agent. The hydrogel is obtained by dissolving silk fibers in a lithium bromide solution and crosslinking through the crosslinking agent. The hydrogel has good elasticity, and can recover more than 90% of its volume/height after being compressed for 100 cycles with a compressive deformation of 20%. The silk is very stable in matrix structure and mechanical properties. After incubation in PBS at 37° C. for 30 days, the content of β-sheets in the secondary structure elements of the silk is less than or equal to 40%, and its compressive modulus is less than or equal to 100% (with a compressive deformation of 20%). The hydrogel has good biocompatibility and adjustable biodegradability, and can be used for repairing or filling tissues in subjects.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57.

### TECHNICAL FIELD

The present disclosure relates to a chemically cross-linked hydrogel and its microspheres, preparation method and application and belongs to the technical field of materials.

### BACKGROUND

Tissue fillers are important materials for repairing congenital tissue defects, traumatic defects and postoperative defects. In recent years, fillings for medical and aesthetic purposes have gradually been widely accepted by the public, followed by a sharp expansion of market demand. However, at present, the injection frequency and cost of tissue fillers are high mainly for the following reasons. The low crosslinking efficiency of hydrogel preparation methods and processes leads to a short in vivo degradation time of the material and a high product production cost. The raw materials qualified to prepare these products are rare and mainly include hyaluronic acid and collagen, of which cross-linked sodium hyaluronate accounts for the vast majority of the market share. In addition, the interface stress of a product hydrogel block is large, which causes a high risk of inflammatory responses. Therefore, the development of a novel efficiently cross-linked hydrogel sphere with a rounded interface and its preparation method is the key to meet the current material demand and reduce cost in the field of tissue fillers.

Raw silk fibers are composed of fibroin (>70%) and sericin proteins (<30%). Degumming process can remove all or a part of sericin from raw silk fibers. The obtained silk fibroin has good biocompatibility, adjustable mechanical properties and adjustable degradation properties. It is widely used in the research of biomedical materials. At present, some products have been approved by FDA and CFDA. The silk described below was silk fibroins regenerated from completely or partially degummed raw silk fibers. The silk can form physically cross-linked hydrogels by simple treatments such as temperature, pH, sonication, shear force, surfactants, and cationic reagents (e.g., Ca²⁺ and K⁺), but the physical crosslinking process is difficult to control, and the hydrogels have high hardness and insufficient toughness. A variety of chemically cross-linked silk hydrogels have been reported, including dityrosine and trityrosine conjugates between tyrosines in silk molecules mediated by superoxide ions produced by horseradish peroxidase (including biomimetic enzymes) catalyzed hydrogen peroxide reaction, ultraviolet light irradiation of riboflavin, and gamma ray irradiation. However, the content of tyrosine in the silk molecule is low (about 5%), which has limited effect on the stability of the secondary structure of the silk molecule. As time goes by, α-helixes and random coils in the secondary structure of the hydrogel are gradually transformed into β-sheets, thus losing the properties of the newly prepared chemically cross-linked hydrogel, such as elasticity and light transmittance. In addition, horseradish peroxidase (including biomimetic enzymes) can cause immune responses and metabolic disorders in the body. Paraformaldehyde can react with the amino group of lysine and the phenolic group of tyrosine in silk molecules to form chemically cross-linked hydrogels, but the cytotoxicity of paraformaldehyde limits its application. Genipin is a natural crosslinking agent that can form chemically cross-linked hydrogels by reacting with lysine and arginine, but lysine and arginine account for a small percentage in the composition of silk, and the content of each amino acid is about 0.6 mol %, resulting in low crosslinking efficiency.

As FDA-approved crosslinking agents, diglycidyl ether crosslinking agents, represented by 1, 4-butanediol diglycidyl ether (BDDE), with low toxicity (relative to divinyl sulfone, DVS) and biodegradability, form stable covalent ether bonds with free hydroxyl and carboxyl groups under certain conditions and are widely used in injectable cross-linked hyaluronic acid hydrogels, as disclosed in, such as, WO2017001056A1 and WO2014206701A1, Dermatol Surg. 2013 December; 39(12): 1758-1766, A Review of the Metabolism of 1,4-Butanediol Diglycidyl Ether—Cross-linked Hyaluronic Acid Dermal Fillers. The patent US20140315828A1 reports that the method of crosslinking HA with BDDE can be used to co-crosslink silk fibroin and HA to form a composite hydrogel, but it is not mentioned that in the absence of HA, BDDE can crosslink silk fibroin molecules to form a hydrogel. Furthermore, reports of using BDDE as a crosslinking agent to crosslink pure silk to form a hydrogel have not been researched by the inventors.

### SUMMARY

In theory, if BDDE can crosslink amino acids containing free hydroxyl groups (serine, threonine, tyrosine, and the like, with a content of about 19.47%), amino acids containing free carboxyl groups (glutamic acid, aspartic acid, and the like, with a content of about 2.52%) and their C-terminal carboxyl groups in silk molecules, BDDE could firmly lock silk molecules to form chemically cross-linked hydrogels, which would be more stable in terms of secondary structure and mechanical properties than gels formed by using about 5% of tyrosine under the action of HRP. This is a very important topic worth exploring for the research and application of silk materials.

In order to solve the above problems, the present disclosure provides a chemically cross-linked silk hydrogel and its microspheres, preparation method and application. The hydrogel of the present disclosure is obtained by chemical reaction between a chemical crosslinking agent and silk, further forming a matrix network between silk molecules through chemical bonds and curing.

The first objective of the present disclosure is to provide a chemically cross-linked hydrogel, which is formed by the reaction of silk with a crosslinking agent, wherein the crosslinking agent is a diglycidyl ether crosslinking agent, and the mass of silk fibroin accounts for 70% or above of the dry weight of the raw material of the silk.

Further, the raw material of the silk is able to dissolve in an aqueous solution of lithium bromide.

Further, the diglycidyl ether crosslinking agent is selected from the group consisting of diglycidyl ether, 1,4-butanediol diglycidyl ether, 1,3-diglycidyl glyceryl ether, bisphenol A diglycidyl ether and its derivatives, resorcinol diglycidyl ether, tris(4-hydroxyphenyl)methane triglycidyl ether, neopentyl glycol diglycidyl ether, and combinations thereof.

The second objective of the present disclosure is to provide a preparation method of the above-mentioned chemically cross-linked hydrogel, comprising the following steps:

S1, dissolving silk fibers in an aqueous solution of lithium bromide to obtain a mixed solution containing silk; and

S2, adding a diglycidyl ether crosslinking agent to the mixed solution obtained in step S1 to carry out a crosslinking reaction to obtain the chemically cross-linked hydrogel.

By adopting the method of the present disclosure, a 100% silk hydrogel cross-linked by a conventional, safe and FDA-approved diglycidyl ether crosslinking agent, such as BDDE, can be obtained, wherein the 100% silk hydrogel means that the raw material in the hydrogel only includes silk solutes and crosslinking agents for crosslinking, without HA and other components that can participate in crosslinking.

Further, subsequent to step S2, the method further includes a step of removing lithium bromide, unreacted chemical crosslinking agent (i.e., the diglycidyl ether crosslinking agent) and free silk.

Further, in step S1, a concentration of lithium bromide in the mixed solution ranges from 1 M to 10 M, preferably from 2 M to 9.8 M.

Further, in step S1, a concentration of silk in the mixed solution ranges from 1 mg/mL to 300 mg/mL, preferably from 10 mg/mL to 250 mg/mL.

Further, a ratio of the mass of the silk to the volume of the diglycidyl ether crosslinking agent is controlled to be 1 g:0.5 μL to 1 mL, preferably, 1 g:0.5 μL to 500 μL.

Further, in step S2, the crosslinking reaction is carried out at a temperature between 10° C. and 100° C., more preferably between 25° C. and 80° C.

Further, in step S2, the crosslinking reaction is carried out for 3 min to 24 h, preferably for 5 min to 12 h, and more preferably for 1 h to 6 h.

Further, in step S1, a mass fraction of silk fibroin in the silk fibers is 70% or above.

Further, a mass fraction of sericin in the silk fibers is less than or equal to 30%.

Further, the silk fibers are able to dissolve in an aqueous solution of lithium bromide.

In the present disclosure, dissolving in an aqueous solution of lithium bromide means forming a uniformly dispersed system without large clusters visible to the naked eye.

In the present disclosure, the silk is degummed silk or genetically engineered silk, and the degummed silk is partially degummed natural silk fibers or completely degummed natural silk fibers, wherein the content of sericin in the silk is less than 30% of the total weight of the silk.

When the degummed silk is completely degummed natural silk, the mixed solution containing silk obtained in S1 does not contain sericin.

When the degummed silk is partially degummed natural silk, the mixed solution containing silk obtained in S1 contains sericin.

When the silk source is genetically engineered silk, the mixed solution containing silk obtained in S1 contains sericin.

In the present disclosure, natural silk refers to silk fibers spit out by silkworms, which can be silk fibers spit out by conventionally cultured silkworms, or silk fibers spit out by silkworms using genetic bioengineering technology (referred to as genetically engineered silk).

The source of regenerated silk described in the present disclosure is natural silk fiber, which is obtained by extracting silk from raw silk fiber materials such as silkworm cocoons using a biochemical engineering technology.

The third objective of the present disclosure is to provide a type of chemically cross-linked hydrogel microspheres, the hydrogel microspheres are formed by the reaction of silk with a crosslinking agent, wherein the crosslinking agent is a diglycidyl ether crosslinking agent.

Further, the diglycidyl ether crosslinking agent is selected from the group consisting of diglycidyl ether, 1,4-butanediol diglycidyl ether, 1,3-diglycidyl glyceryl ether, bisphenol A diglycidyl ether and its derivatives, resorcinol diglycidyl ether, tris(4-hydroxyphenyl)methane triglycidyl ether, neopentyl glycol diglycidyl ether, and combinations thereof.

Further, the chemically cross-linked hydrogel microspheres have a particle size ranging from 50 μm to 300 μm, further from 100 μm to 300 μm.

The fourth objective of the present disclosure is to provide a preparation method of the above-mentioned chemically cross-linked hydrogel microspheres, comprising the following steps:

S01, dissolving silk fibers in an aqueous solution of lithium bromide to obtain a mixed solution containing silk; and

S02, adding a diglycidyl ether crosslinking agent to the mixed solution obtained in step S01, and well mixing the resulting solution to obtain a reaction solution; and

S03, adding into an oil phase system the reaction solution obtained in step S02, and stirring the mixture to carry out a crosslinking reaction to obtain the chemically cross-linked hydrogel microspheres.

Further, subsequent to step S03, the method further includes a step of removing lithium bromide, unreacted chemical crosslinking agent and free silk.

Further, a concentration of lithium bromide in the mixed solution ranges from 1 M to 10 M, preferably from 2 M to 9.8 M.

Further, a concentration of silk in the mixed solution is 1 mg/mL to 300 mg/mL, preferably 10 mg/mL to 250 mg/mL.

Further, a ratio of the mass of the silk to the volume of the chemical crosslinking agent is controlled to be 1 g:0.5 μL-1 mL, preferably, 1 g:0.5 μL to 500 μL.

Further, in step S03, the crosslinking reaction is carried out at a temperature between 10° C. and 100° C., more preferably between 25° C. and 80° C.

Further, in step S03, the crosslinking reaction is carried out for 3 min to 24 h, preferably for 5 min to 12 h, and more preferably for 5 min to 6 h.

Further, in step S01, a mass fraction of silk fibroin in the silk fibers is 70% or above.

Further, in step S03, a volume ratio of the oil phase system to the reaction solution is greater than 1:1, and the stirring speed ranges from 100 rpm to 15000 rpm. Preferably, the volume ratio of the oil phase system to the reaction solution is 2:1 to 500:1.

Further, in step S03, the oil phase system includes but is not limited to: an animal oil, such as fatty acids; a vegetable oil, such as soybean oil, castor oil, corn oil, or the like; a mineral oil, such as petrolatum, paraffin, ozokerite, or the like; or an organic solvent, such as chloroform, ethyl acetate, or acetonitrile.

The fifth objective of the present disclosure is to provide application of the chemically cross-linked hydrogel and/or the chemically cross-linked hydrogel microspheres in tissue engineering filling, repair and/or drug delivery.

Further, the application includes the application in preparation of a composition for arthritis treatment, medical cosmetic surgery or ophthalmic disease treatment.

The method for treating ophthalmic diseases of the present disclosure includes injecting administration into the eyeball and applying lubricating drops on the surface of the eyeball.

The arthritis treatment of the present disclosure includes intra-articular injection administration, so as to achieve lubrication in the joint cavity, and have a certain repairing effect on cartilage and bone tissue.

The medical cosmetic surgery of the present disclosure includes tissue filling and tissue repair.

Further, a volume fraction of hydrogel particles and/or chemically cross-linked hydrogel microspheres obtained from the chemically cross-linked hydrogel in the composition ranges from 50% to 100%, and the mass fraction of the silk ranges from 5% to 20%.

Further, the composition further includes one or more of a stabilizer, a lubricant and an osmotic pressure regulator.

In the present disclosure, the stabilizer is selected from the group consisting of hyaluronic acid, cellulose, polyethylene glycol, polyvinyl alcohol, mannitol, glycerin, and the like.

In the present disclosure, the lubricant is selected from the group consisting of hyaluronic acid, polyethylene glycol, propylene glycol, phospholipids, liposomes, an oily substance (such as mineral oil, corn oil, soybean oil and the like), collagen, chitosan, cellulose, and the like.

In the present disclosure, the osmotic pressure regulator is selected from the group consisting of mannitol, sorbitol, glycerol, sodium chloride, glucose and the like.

Further, the composition also comprises one or more of a bioactive reagent, an extracellular matrix, a cell and a drug.

In the present disclosure, the bioactive reagent is selected from the group consisting of hydroxyapatite, a cytokine, a growth factor, a peptide, a mimic peptide, an antibody, a nucleic acid substance, a cell, and the like.

In the present disclosure, the drug is selected from the group consisting of a therapeutic agent, a nutritional agent, an anesthetic agent, an anti-inflammatory and analgesic agent, an antibiotic agent, and the like.

The functional component (one or more of the stabilizer, the lubricant, the osmotic pressure regulator, the bioactive reagent, the extracellular matrix, the cell, and the drug) carried in the hydrogel of the present disclosure can be preloaded (blended with silk before the reaction), or post-loaded (loaded into the hydrogel by osmotic adsorption after reaction), or both pre-loaded and post-loaded.

The sixth objective of the present disclosure is to provide application of the chemically cross-linked hydrogel and/or the chemically cross-linked hydrogel microspheres in preparation of thin films, scaffolds or hard bone materials.

The seventh objective of the present disclosure is to provide a hydrogel sphere composition, wherein a volume fraction of the hydrogel particles and/or hydrogel microspheres in the composition ranges from 50% to 100%, and a mass fraction of silk is 5% to 20%, wherein the hydrogel particles and/or the hydrogel microspheres are formed by the reaction of silk with a crosslinking agent, and the crosslinking agent is a diglycidyl ether crosslinking agent.

The eighth objective of the present disclosure is to provide application of the above-mentioned hydrogel spheres composition in the above-mentioned fields.

The beneficial effects of the present disclosure are as follows:

(1) For the secondary structure of the silk in the silk hydrogel of the present disclosure, the content of β-sheets determined by infrared spectroscopy is less than or equal to 32%. The hydrogel has good elasticity, and can recover more than 90% of its volume/height after being compressed for 100 cycles with a compressive deformation of 20%. The matrix structure and mechanical properties of silk are very stable. After incubation in PBS at 37° C. for 30 days, the content of β-sheets in the secondary structure elements of the silk is less than or equal to 40%, and the compressive modulus is increased by one-fold or less (with a compressive deformation of 20%). The silk hydrogel of the present disclosure has the above-mentioned excellent mechanical properties mainly because when the crosslinking reaction of the present disclosure occurs, there are hydroxyl-containing amino acids (serine, threonine, tyrosine, and the like, with a content of about 19.47% of the total amino acids) and carboxyl-containing amino acids (glutamic acid, aspartic acid, and the like, with a content of 2.52% of the total amino acids) as reaction sites, multiple reaction sites can be cured by forming a matrix network through chemical bonds, and the formed structure is more stable than structures formed by conventional methods.

(2) The hydrogel of the present disclosure has good biocompatibility and adjustable biodegradability, and can be used for repairing or filling tissues in subjects.

(3) In the reaction system composed of the silk, the lithium bromide and the crosslinking agent of the present disclosure can react in water-in-oil dispersion system to form hydrogel microspheres of uniform size, and the hydrogel microspheres are further improved in terms of injectability, inflammatory response induced by foreign substances, in vivo degradability, induction of collagen deposition, and structural stability of an implant. Based on the lubricating properties of the hydrogel spheres (i.e., the viscoelasticity and toughness of the sphere itself), the hydrogel spheres can act on an object interface through a principle of “bearing balls” to promote lubrication. The hydrogel microspheres of the present disclosure can be applied to the human body, can lubricate interfaces of bone and cartilage, the surface of the cornea, and the like and can be used as a component of joint cavity injections and eye drops for the treatment and repair of arthritis and dry eye diseases. The hydrogel of the present disclosure has good biocompatibility, adjustable mechanical properties and structural stability.

### BRIEF DESCRIPTION OF THE DRAWINGS

FIG. 1 shows the crosslinking and reaction efficiency of silk in different silk-dissolving systems. A: the crosslinking of silk in different silk-dissolving systems; B: the reaction efficiency of silk in different silk-dissolving systems.

FIG. 2 shows the effect of the concentration of degummed silk fibers on BDDE-cross-linked silk. A: reaction efficiency of hydrogels with different concentrations of silk; B: compressive modulus of hydrogels with different concentrations of silk; C: OD value curves at 400-700 nm; D: OD values at 550 nm; E: photographic images of hydrogels with different concentrations of silk.

FIG. 3 shows the effect of the concentration of degummed silk fibers on the microstructure of BDDE-cross-linked silk. A: 250 mg/mL; B: 200 mg/mL; C: 150 mg/mL; D: 100 mg/mL; E: 75 mg/mL.

FIG. 4 shows the effect of the amount of BDDE on BDDE-cross-linked silk. A: reaction efficiency of hydrogels with different silk/BDDE ratios; B: compressive modulus of hydrogels with different silk/BDDE ratios; C: OD value curves of hydrogels with different silk/BDDE ratios at 400-700 nm; D: OD values of hydrogels with different silk/BDDE ratios at 550 nm; E: photographic images of hydrogels with different silk/BDDE ratios.

FIG. 5 shows the effect of the amount of BDDE added on the microstructure of BDDE-cross-linked silk. A: 1 g:32.5 μL, B: 1 g:62.5 μL, C: 1 g:125 μL, D: 1 g:200 μL, E: 1 g:250 μL, F: 1 g:350 μL, G: 1 g:500 μL.

FIG. 6 shows the stability evaluation of BDDE-cross-linked silk in 9.3M lithium bromide solution.

FIG. 7 shows the in vitro degradation of BDDE-cross-linked silk hydrogels. a: silk/BDDE hydrogel in a solution containing protease XIV; b: silk/BDDE hydrogel in PBS buffer, pH7.4; c: silk/sonication hydrogel in a solution containing protease XIV; d: silk/sonication hydrogel in PBS buffer, pH7.4; e: silk/HRP hydrogel in a solution containing Protease XIV; f: silk/HRP hydrogel in PBS buffer, pH7.4.

FIG. 8 shows the biosafety evaluation of BDDE-cross-linked silk hydrogels by using in vitro cell culture. A: the morphology of rat bone marrow mesenchymal stem cells cultured on different hydrogels on day 5 and day 7; B: the proliferation fold diagram of stem cells cultured on different hydrogels.

FIG. 9 shows the water contact angles of BDDE-cross-linked silk hydrogels. A: photographic images showing the water drops contacting the surface of different hydrogels. Silk/sonication hydrogel (a), silk/BDDE hydrogel (b); B: trend of the dynamic change of water contact angles of hydrogels; C: comparison of initial contact angles of hydrogels.

FIG. 10 shows the biosafety evaluation of BDDE-cross-linked silk hydrogels by animal studies. A: H&E staining of silk/BDDE hydrogel; B: H&E staining of silk/sonication hydrogel.

FIG. 11 shows the morphology of chemically cross-linked silk hydrogel spheres.

FIG. 12 shows the biosafety of subcutaneous injection of chemically cross-linked silk hydrogel spheres (H&E staining).

FIG. 13 shows the biosafety of subcutaneous injection of chemically cross-linked silk hydrogel spheres (MASSON staining).

FIG. 14 shows the pain threshold in knee joint of the OA rat model at 1st, 2nd, 3rd, 4th, 5th, and 6th weeks after the establishment of OA model.

### DETAILED DESCRIPTION OF THE PREFERRED EMBODIMENT

The present disclosure will be further described below with reference to the accompanying specific embodiments and accompanying drawings, so that those skilled in the art can better understand the disclosure and implement it, but the embodiments given here are not intended to limit the present disclosure.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

### Example 1

Sixty g of raw silk fibers and 25.44 g of anhydrous sodium carbonate were weighed for use, and 12 L of deionized water was taken and poured into a stainless steel bucket and heated with an induction cooker. The weighed anhydrous sodium carbonate was added to the deionized water when the deionized water was about to boil. The mixture was further heated and stirred until boiling so that the anhydrous sodium carbonate was fully dissolved. The weighed raw silk fibers were then added to the solution and the resulting mixed solution was kept boiling for 30 min and stirred every 5 min to dissolve the sericin on the surface of the raw silk fibers. The degummed raw silk fibers were rubbed four times with deionized water so that the sericin on the surface of the raw silk fibers was removed thoroughly. Finally, the degummed silk fibers were wrung out and dried in a fume hood overnight. As reported, the removal rate of sericin under this condition is 100%.

Ten g of the dried degummed silk fibers were taken and placed into a container with 40 mL of 9.3 M lithium bromide and the mixture was then stirred with a glass rod. The lithium bromide solution and the silk fibers were mixed and heated in an oven with a temperature of 60° C. for 4 h to accelerate the dissolution of the silk fibers. Next, the completely dissolved silk solution was poured into a dialysis bag (with MWCO of 3500 Da) and sealed. The dialysis bag containing the silk solution was placed into a container containing 5 L of deionized water, and the solution was then continuously stirred with a magnetic stirrer at the bottom of the container to dilute the leached lithium bromide. The dialysis was carried out for 3 days, and the water was changed for a total of 7 to 8 times. After the desalting was complete, the silk solution was placed in a centrifuge bottle and centrifuged twice at 9000 rpm and a low temperature of 4° C., and finally a clear silk solution was obtained and then stored in a refrigerator with a temperature of 4° C.

The concentration of the silk solution was further determined by a weighing method as follows: the weight of a weighing dish was measured and denoted W; 1 mL of the silk solution was then added to the dish and the weight of the dish containing the solution was measured and denoted W₁; after being dried in an oven for 24 h, the dish containing the silk solution was weighed again and the result was denoted W₂. The concentration (w/w) of the silk solution was obtained according to the following formula.

Concentration=(W₁−W₂)/W₁×100%

a. Silk-dissolving system using lithium bromide: 10 mL of 9.3 M lithium bromide was taken to thoroughly soak and dissolve 1.5 g of degummed silk fibers at 60° C. for 1.5 h to obtain a lithium bromide silk-dissolving solution with a concentration of 150 mg/mL. 400 μL of butanediol diglycidyl ether (BDDE) was then added to the silk-dissolving solution. The resulting solution was mixed thoroughly and then set still for 3 h in an oven with a temperature of 60° C. to have a reaction.

b. Silk-dissolving system using calcium chloride/absolute ethanol/ultrapure water: 111 g of calcium chloride, 92.12 mL of absolute ethanol, and 114 mL of ultrapure water were mixed well to obtain a ternary mixed solution. 10 mL of the ternary mixed solution was taken to thoroughly soak and dissolve 1.5 g of degummed silk fibers at 80° C. for 1.5 h to obtain a ternary mixed silk-dissolving solution with a concentration of 150 mg/mL. 400 μL of BDDE was then added to the silk-dissolving solution. The resulting solution was mixed thoroughly and then set still for 3 h in an oven with a temperature of 60° C. to have a reaction.

c. Silk-dissolving system using formic acid/calcium chloride: 4 g of calcium chloride was dissolved in 100 mL of formic acid to obtain a formic acid/calcium chloride mixed solution. 10 mL of the formic acid/calcium chloride mixed solution was taken to thoroughly soak and dissolve 1.5 g of degummed silk fibers at room temperature for 1.5 h to obtain a formic acid/calcium chloride silk-dissolving solution with a concentration of 150 mg/mL. 400 μL of BDDE was then added to the silk-dissolving solution. The resulting solution was mixed thoroughly and then set still for 3 h in an oven with a temperature of 60° C. to have a reaction.

d. Silk-dissolving system using formic acid/lithium bromide: 1 mL of formic acid and 13.3 mL of 8 M lithium bromide were mixed well to obtain a formic acid/lithium bromide mixed solution. 10 mL of the formic acid/lithium bromide mixed solution was taken to thoroughly soak and dissolve 1.5 g of degummed silk fibers at room temperature for 1.5 h to obtain a formic acid/lithium bromide silk-dissolving solution with a concentration of 150 mg/mL. 400 μL of BDDE was then added to the silk-dissolving solution. The resulting solution was mixed thoroughly and then set still for 3 h in an oven with a temperature of 60° C. to have a reaction.

e. An aqueous solution of regenerated silk stored in a refrigerator with a temperature of 4° C. and air-dried and concentrated to 150 mg/mL was taken to react with 400 μL of BDDE and the reaction system was mixed thoroughly and then set still for 3 h in an oven with a temperature of 60° C. for gelation.

f. Alkaline reaction system: 1.25 mL of 1 M sodium hydroxide was added to an aqueous solution of regenerated silk stored in a refrigerator with a temperature of 4° C. and air-dried and concentrated to 200 mg/mL to obtain a mixed solution in which the concentration of silk was 150 mg/mL and the concentration of sodium hydroxide was 0.25 M. 400 μL of BDDE was then added to the mixed solution. The resulting solution was mixed thoroughly and then set still for 3 h in an oven with a temperature of 60° C. to have a reaction.

g. Under a lithium bromide reaction system, using a regenerated silk source: lithium bromide powder was added to an aqueous solution of regenerated silk stored in a refrigerator with a temperature of 4° C. and air-dried and concentrated to 200 mg/mL to obtain a mixed solution in which the concentration of silk was 150 mg/mL and the concentration of lithium bromide is 9.3 M. 400 μL of BDDE was then added to the mixed solution. The resulting solution was mixed thoroughly and then set still for 3 h in an oven with a temperature of 60° C. to have a reaction.

h. Under a lithium bromide reaction system, using silk that was obtained by dissolving silk fibers in the ternary mixed solution: 111 g of calcium chloride, 92.12 mL of absolute ethanol, and 114 mL of ultrapure water were mixed well to obtain a ternary mixed solution. 10 mL of the ternary mixed solution was taken to thoroughly soak and dissolve 2 g of degummed silk fibers at 60° C. for 1.5 h to obtain a ternary mixed silk-dissolving solution with a concentration of 200 mg/mL. Lithium bromide powder was added to the ternary mixed silk-dissolving solution to obtain a solution in which the concentration of silk was 150 mg/mL and the concentration of lithium bromide was 9.3 M. 400 μL of BDDE was then added to the mixed solution. The resulting solution was mixed thoroughly and then set still for 3 h in an oven with a temperature of 60° C. to have a reaction.

The reaction system was observed to check whether there was a bulk hydrogel formed therein. The bulk hydrogel was collected, washed with water, freeze-dried and weighed, to calculate the reaction efficiency.

Reaction efficiency=W₂/W₁*100%

Where W₂ represents the weight of freeze-dried hydrogel, and W₁ represents the weight of silk added to the reaction system before crosslinking.

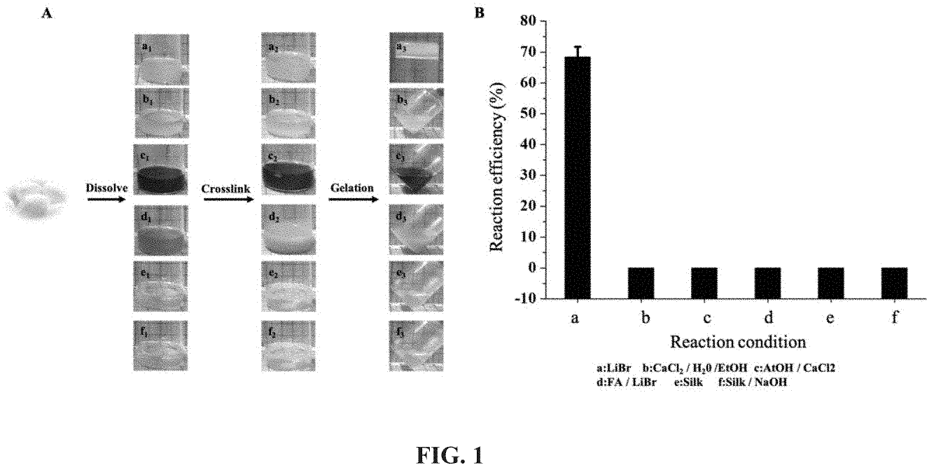

Groups a, b, c, and d were the reported silk-dissolving systems for dissolving degummed silk fibers; Group e was a blank control group; Group f was a reported alkaline condition under which BDDE reacts with hyaluronic acid or hyaluronic acid and regenerated silk to form a hydrogel; Group g was under a lithium bromide reaction system, using a regenerated silk source; Group h was under a lithium bromide reaction system, using the silk source obtained by dissolving silk fibers in the ternary mixed solution. The results of Groups a to f were shown in FIG. 1.

It could be seen from FIG. 1 that only in the 9.3 M lithium bromide silk-dissolving system, the silk was cross-linked to form bulk hydrogel, but no bulk hydrogel was formed in other silk-dissolving systems. It can be concluded that using degummed silk fibers as a silk source, BDDE has specificity in chemical crosslinking with silk in the lithium bromide silk-dissolving system to form a chemically cross-linked pure silk hydrogel.

In addition, according to the method of patent US20140315828A1, the team of inventors found that BDDE cannot crosslink pure regenerated silk to form a hydrogel in an alkaline environment (0.25 M Sodium hydroxide solution).

The inventors conducted experiments on one of or various combinations of diglycidyl ether substances, such as diglycidyl ether, 1,4-butanediol diglycidyl ether (BDDE), 1,3-diglycidyl glyceryl ether, bisphenol A diglycidyl ether and its derivatives, resorcinol diglycidyl ether, tris(4-hydroxyphenyl)methane triglycidyl ether, neopentyl glycol diglycidyl ether, and the like, according to the experimental protocol of Example 1, and obtained experimental phenomena and conclusions similar to those in Example 1.

### Example 2

The inventors conducted orthogonal tests on the residual sericin of degummed silk, the concentration of lithium bromide, lithium bromide silk-dissolving bath ratio, the amount of BDDE, reaction temperature and reaction time.

The sericin residue of degummed silk: 100% (the content of sericin: 30%), 70%, 60%, 50%, 40%, 30%, 20%, 10%, 5%, and 0%.

The concentration of lithium bromide during reaction: 1 M, 2 M, 3 M, 4 M, 5 M, 6 M, 7 M, 8 M, 9.3 M, 9.5 M, and 9.8 M.

Lithium bromide silk-dissolving bath ratio: 0.5 mg/mL, 1 mg/mL, 4 mg/mL, 7 mg/mL, 10 mg/mL, 15 mg/mL, 20 mg/mL, 25 mg/mL, 50 mg/mL, 75 mg/mL, 100 mg/mL, 120 mg/mL, 125 mg/mL, 150 mg/mL, 175 mg/mL, 200 mg/mL, 250 mg/mL, and 300 mg/mL.

The silk/BDDE ratio: 1 g:0.2 μL, 1 g:0.5 μL, 1 g:0.75 μL, 1 g:1 μL, 1 g:2 μL, 1 g:5 μL, 1 g:10 μL, 1 g:20 μL, 1 g:50 μL, 1 g:100 μL, 1 g:120 μL, 1 g:150 μL, 1 g:300 μL, 1 g:500 μL, 1 g:750 μL, and 1 g:1000 μL.

Reaction temperature: −20° C., 0° C., 4° C., 25° C., 37° C., 60° C., and 80° C.

Reaction time: 1 min, 3 min, 5 min, 7 min, 10 min, 20 min, 30 min, 40 min, 1 h, 1.5 h, 3 h, 6 h, 12 h, 18 h, 24 h, 36 h, and 48 h.

(1) In the case with the concentration of lithium bromide, the silk/BDDE ratio, the reaction temperature and the reaction time of the reaction system unchanged, but the concentration of silk changed, the inventors enumerated the following experiments. 2.5 g, 2 g, 1.5 g, 1 g, and 0.75 g of degummed silk fibers were respectively dissolved in 10 mL of 9.3 M lithium bromide (250 mg/mL, 200 mg/mL, 150 mg/mL, 100 mg/mL, and 75 mg/mL), the resulting solutions were incubated in an oven with a temperature of 60° C. for 1 h, and 400 μL of BDDE was added to the reaction systems and the reaction systems were then placed in an oven with a temperature of 60° C. to react for 3 h. The reaction systems were then observed to check whether there was a bulk hydrogel formed therein. The bulk hydrogel was collected, washed with water, freeze-dried and weighed to calculate the crosslinking efficiency.

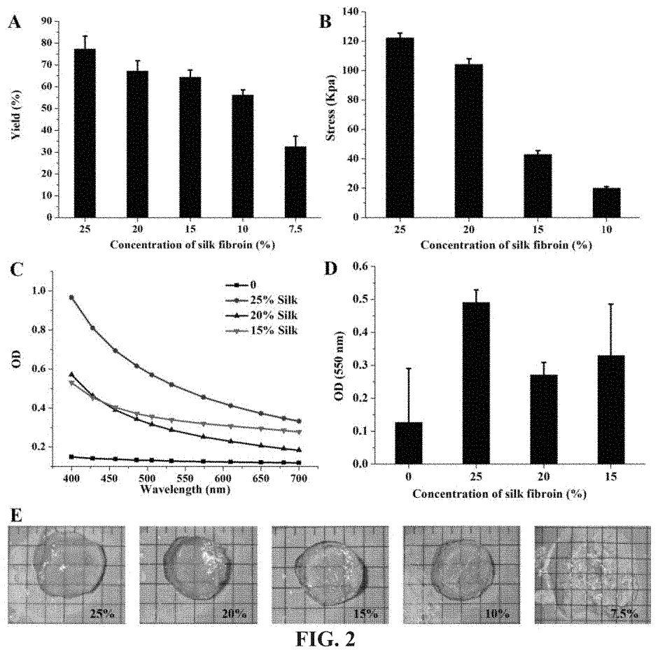

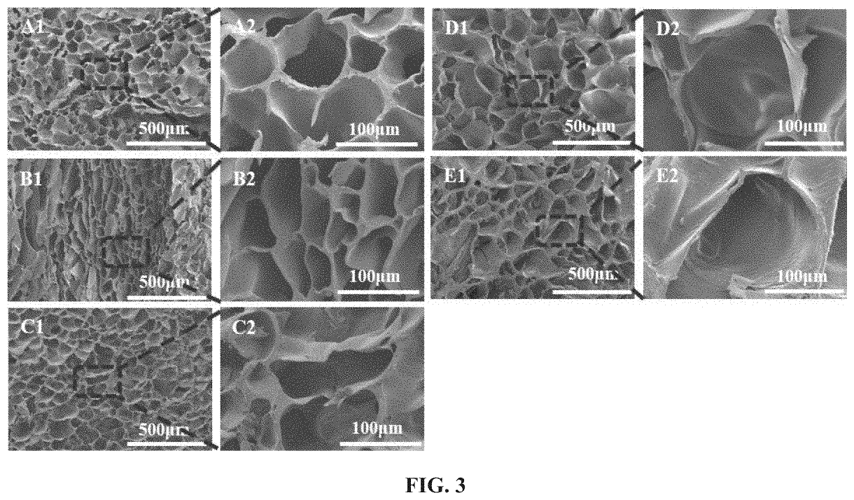

Crosslinking efficiency=W₂/W₁*100%

Where W₂ represents the weight of freeze-dried bulk hydrogel, and W₁ represents the weight of silk added to the reaction system before crosslinking. It could be seen from FIG. 2 that when the reaction concentration of silk was 250 mg/mL, the yield was the highest, reaching 77.3±5.9%; when the reaction concentration was between 200 mg/mL and 100 mg/mL, the difference in yield was not obvious and between 56.11±2.45 and 67.17±4.72%; and when the reaction concentration was 75 mg/mL, the yield was relatively low, about 30%. When the concentration of silk was between 100 mg/mL and 250 mg/mL, the compressive modulus was in the range of 19±1 kPa to 122±3 kPa, and when the concentration of silk was 250 mg/mL, the compressive modulus was the highest, reaching 122±3 kPa. It could be seen from the photos of hydrogels that the BDDE-cross-linked silk was colorless and transparent, which was obviously different from the white color of the physically cross-linked silk. Subsequently, the inventors studied the light transmittance of the BDDE-cross-linked hydrogels. It was found that the light transmittance was poor when the reaction concentration was 250 mg/mL, which was caused by the incompletely dissolved degummed silk fibers remaining in the hydrogel, and this will bring some adverse effect on ophthalmic applications which have requirements on the light transmittance of hydrogels. Light transmittance was very good when the reaction concentration was 200 mg/mL and below. Considering the reaction yield and the light transmittance of the hydrogels, the optimal reaction concentration is 200 mg/mL. In the range of 1 mg/mL to 300 mg/mL, ideal silk hydrogels can be obtained. In the range of 10 mg/mL to 250 mg/mL, more excellent silk hydrogels can be obtained.

The microstructure of BDDE-cross-linked hydrogels was studied. The above freeze-dried hydrogels were sprayed with gold and observed by SEM, as shown in FIG. 3. It was found that the pore size of the hydrogels increased with the decrease of the concentration of silk; the proportion of pores larger than 100 μm also increased from 32% to 83% with the decrease of the concentration of silk; when the concentrations of silk were 10 mg/mL and 7.5 mg/mL, the mean pore size inside the hydrogels reached 200 μm. The pore size structure of the silk hydrogel of the present disclosure is larger than that of the hydrogel prepared by the reported equivalent concentrations of fibroin, which is very important for the research and application of the hydrogel in the field of tissue regeneration materials.

(2) In the case with the concentration of lithium bromide, the concentration of silk, the reaction temperature and the reaction time of the reaction system unchanged, but the silk/BDDE ratio changed, the inventors enumerated the following experiments. 2 g of degummed silk fibers were respectively dissolved in 10 mL of 9.3 M lithium bromide, the resulting solutions were incubated in an oven with a temperature of 60° C. for 1 h, and 65 μL, 125 μL, 250 μL, 400 μL, 500 μL, 700 μL, and 1000 μL of BDDE were respectively added to the reaction system and in the obtained reaction solution, the silk/BDDE ratios were 1 g:32.5 μL, 1 g:62.5 μL, 1 g:125 μL, 1 g:200 μL, 1 g:250 μL, 1 g:350 μL, and 1 g:500 μL, respectively. The reaction systems were then placed in an oven with a temperature of 60° C. to react for 3 h. The reaction systems were then observed to check whether there was a bulk hydrogel formed therein. The bulk hydrogel was collected, washed with water, freeze-dried and weighed to calculate the crosslinking yield.

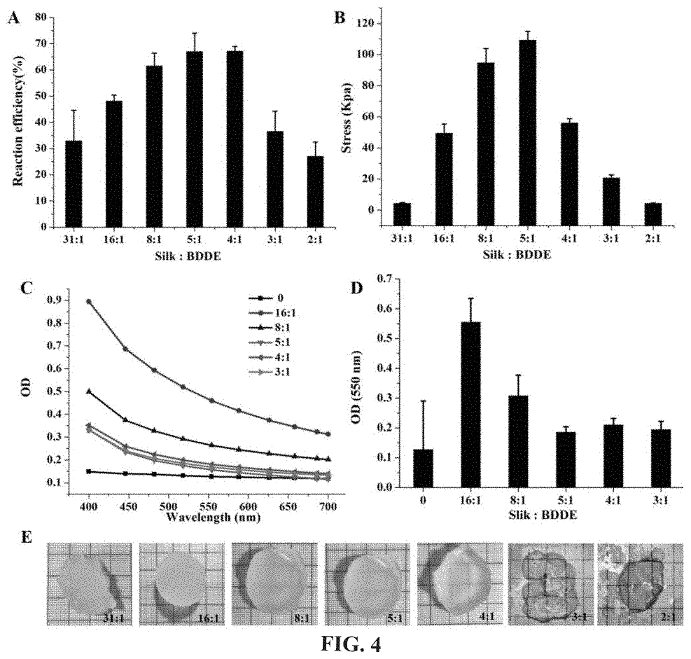

Yield=W₂/W₁*100%

Where W₂ represents the weight of freeze-dried hydrogel, and W₁ represents the weight of silk added to the reaction system before crosslinking. It could be concluded from FIG. 4 that the yield increased first and then decreased with the increase of the amount of BDDE. When the amount of BDDE increased according to the silk/BDDE ratio increasing from 1 g:32.5 μL to 1 g:250 μL, the yield increased from 30% to 67±7%. As the further increase of the amount of the crosslinking agent BDDE, the yield did not increase any longer, but decreased with the increase of the amount of BDDE. The compressive modulus of the hydrogel had the same change trend as the yield. The compressive modulus was within the range of 4±0.3 kPa to 109±5 kPa. When silk: BDDE=1 g:200 μL (W/W), the compressive modulus was the highest, reaching 109±5 kPa. Subsequently, the inventors studied the light transmittance of the BDDE-cross-linked hydrogels. It was found that the light transmittance of the silk hydrogel increased with the increase of the amount of BDDE, and the best light transmittance was achieved when the ratio was 1 g:200 μL. Considering the reaction yield and the light transmittance of the hydrogel, the inventors believe that when the amount of BDDE increases according to the silk/BDDE ratio increasing from 1 g:32.5 μL to 1 g:200 μL, the crosslinking efficiency of silk increases with the increase of the amount of BDDE. When the amount of BDDE is less than that corresponding to the ratio of 1 g:200 μL, part of the uncross-linked silk is encapsulated in the chemically cross-linked hydrogel to form a physically cross-linked hydrogel, which reduces the light transmittance of the hydrogel. However, the crosslinking efficiency of silk decreases when the amount of BDDE is further increased. When the reaction concentration of silk is 200 mg/mL, the optimal reaction condition is the amount of BDDE corresponding to the silk/BDDE ratio of 1 g:200 μL. In the range of 1 g:0.5 μL to 1000 μL, ideal silk hydrogels can be obtained. In the range of 1 g:0.5 μL to 500 μL, silk hydrogels with more excellent performance can be obtained.

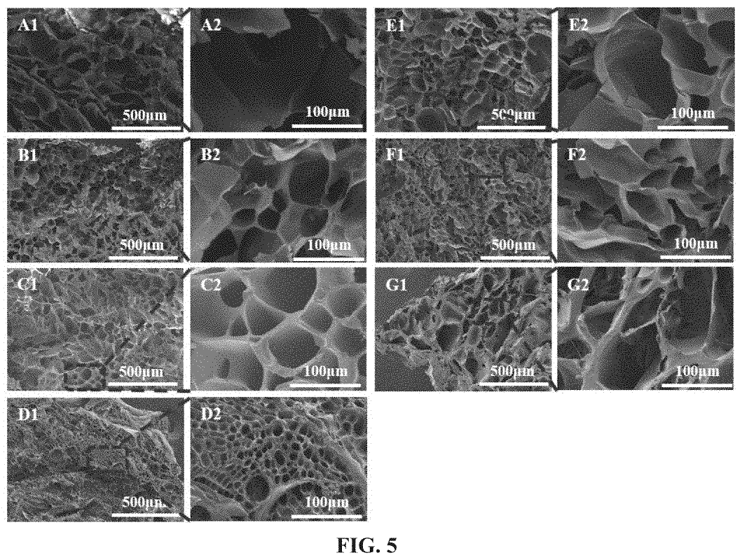

The microstructure of BDDE-cross-linked hydrogels was studied. The above freeze-dried hydrogels were sprayed with gold and observed by SEM, as shown in FIG. 5. It was found that the pore size of the hydrogels first decreased and then increased with the increase of the amount of BDDE, and when the ratio was 1 g:200 μL, the pore size was the smallest. Considering the above yield results, it is further proved that when the ratio is 1 g:200 μL, both the crosslinking efficiency of silk and the hydrogel density reach the highest. In the range of 1 g:0.5 μL to 1000 μL, ideal silk hydrogels can be obtained. In the range of 1 g:0.5 μL to 500 μL, silk hydrogels with more excellent performance can be obtained.

(3) In the case with the concentration of lithium bromide, the concentration of silk, the silk/BDDE ratio, and the reaction temperature of the reaction system unchanged, but the reaction time changed, the inventors enumerated the following experiments. Four 50 mL centrifuge tubes were prepared, each added with 10 mL of 9.3 M lithium bromide. 2 g of degummed silk fibers was then added to each of the four centrifuge tubes and thoroughly soaked and dissolved at 60° C. for 1.5 h to obtain a lithium bromide silk-dissolving solution with a concentration of 200 mg/mL. 400 μL of BDDE was then added to each of the four centrifuge tubes and the resulting solutions were mixed thoroughly and then set still for 1 h, 3 h, 6 h, and 24 h in an oven with a temperature of 60° C., respectively. The gelation state of each group was observed and the yield and compressive modulus were determined after dialysis.

It was found that when the crosslinking time was 1 h, 3 h, 6 h, and 24, there was no significant difference in the hydrogels of each group, and all of them were colorless, transparent and had formability to some degree. The hydrogel yield did not change obviously with increase of crosslinking time. When the crosslinking time was 1 h, the hydrogel yield was 67.78±8.76%. When the crosslinking time was 3 h, the hydrogel yield was 70.77±9.82%. When the crosslinking time was 6 h, the hydrogel yield was 68.99±3.24%. When the crosslinking time was 24 h, the hydrogel yield was 65.99±5.57%. There was also no significant difference in the compressive modulus of the hydrogels with different crosslinking times. When the crosslinking time was 1 h, the compressive modulus of the hydrogel was 102.30±2.17 kPa. When the crosslinking time was 3 h, the compressive modulus of the hydrogel was 108.98±5.16 kPa. When the crosslinking time was 6 h, the compressive modulus of the hydrogel was 99.79±3.66 kPa. When the crosslinking time was 24 h, the compressive modulus of the hydrogel was 104.37±5.27 kPa. Considering the test results of yield and compressive modulus, it can be seen that with the increase of the crosslinking time, the differences in the yield and compressive modulus of the hydrogel are not obvious. The crosslinking is completed when the crosslinking time is 1 h, but the yield and compressive modulus both reach the highest when the crosslinking time is 3 h. In the range of 3 min to 24 h, ideal silk hydrogels can be obtained. In the range of 3 min to 12 h, silk hydrogels with more excellent performance can be obtained.

(4) In the case with the reaction time, the concentration of silk, the reaction temperature, and the silk/BDDE ratio were unchanged, but the concentration of lithium bromide of the reaction system changed, the inventors enumerated the following experiments. 50 mL of 9.3 M lithium bromide was added to a 100 mL beaker. 12.5 g of degummed silk fibers were then added to the beaker and thoroughly soaked and dissolved at 60° C. for 1.5 h to obtain a 25 wt % lithium bromide silk-dissolving solution. Lithium bromide solutions with different concentrations were prepared respectively, a certain amount of 25 wt % lithium bromide silk-dissolving solution was added in 50 mL centrifuge tubes, and then lithium bromide solutions with different concentrations in certain amount were added to the centrifuge tubes respectively. The resulting solutions were mixed thoroughly, thus obtaining 5 parts of lithium bromide silk-dissolving solution in which the concentrations of silk were 15 wt % and the concentrations of lithium bromide were 5.58 M, 6.51 M, 7.44 M, 8.37 M and 9.3 M, respectively. 400 μL of BDDE was then added for reaction, and the reaction solutions were mixed thoroughly and then set still in an oven with a temperature of 60° C. for 3 h for gelation. In the present disclosure, 250 mg/mL silk-dissolving solution was used as the mother solution of silk, and the concentration of silk being 150 mg/mL was adopted as the reaction concentration, so that the influence of lithium bromide concentration on the chemical crosslinking efficiency can be studied in a wider range. In the silk-dissolving solution using lithium bromide, the concentration of lithium bromide can be changed to prepare silk hydrogels under the condition of different concentrations of lithium bromide solution. The gelation state of each group was observed and the yield and compressive modulus were determined after dialysis.

It was found that the hydrogels were all colorless and transparent. When the concentration of lithium bromide were 9.3 M, 8.37 M, 7.44 M, 6.51 M, and 5.58 M, the hydrogels had certain formability. The hydrogel yield decreased with decrease of the concentration of lithium bromide. When the concentration of lithium bromide was 9.3 M, the yield was 65.44±8.01%. When the concentration of lithium bromide was 8.37 M, the yield was 52.31±3.53%. When the concentration of lithium bromide was 7.44 M, the yield was 48.11±4.21%. When the concentration of lithium bromide was 6.51 M, the yield was 31.22±5.14%. When the concentration of lithium bromide was 5.58 M, the yield was 20.34±2.4%. The highest yield was obtained when the concentration of lithium bromide was 9.3 M. The compressive modulus of the hydrogels in the case of different concentrations of lithium bromide had the same change trend with the yield, and decreased with the decrease of the concentration of lithium bromide. The compressive modulus was 42.92±1.26 kPa when the concentration of lithium bromide was 9.3 M. The compressive modulus was 14.63±0.97 kPa when the concentration of lithium bromide was 8.37 M. The compressive modulus was 11.39±0.70 kPa when the concentration of lithium bromide was 7.44 M. The compressive modulus was 8.98±0.57 kPa when the concentration of lithium bromide was 6.51 M. The compressive modulus was 6.71±0.43 kPa when the concentration of lithium bromide was 5.58 M. The compressive modulus of the hydrogel reached the highest when the concentration of lithium bromide was 9.3 M. Considering the test results of yield and compressive modulus, it can be seen that when the concentration of lithium bromide is 9.3 M, the hydrogel with the best morphology is obtained, and the compressive modulus and yield are both the highest. In the range of 1 M to 10 M, ideal silk hydrogels can be obtained. In the range of 2 M to 9.8 M, silk hydrogels with more excellent performance can be obtained.

According to the experimental scheme of Example 2, the inventors conducted research on the residual sericin of degummed silk, the concentration of lithium bromide in the reaction system, the total mass fraction of silk, the ratio of the total mass of silk to the volume of the crosslinking agent, reaction temperature and reaction time. It was found that under the condition that the residual sericin is less than or equal to 30%, the concentration of lithium bromide in the reaction system was greater than or equal to 1 M, the total mass fraction of silk was within a range of 1 mg/mL to 300 mg/mL, the ratio of the total mass of silk to the volume of the crosslinking agent was within a range of 1 g:5 μL to 1 mL, the reaction temperature was within a range of 25° C. to 80° C., and the reaction time was within a range of 3 min to 24 h, the hydrogels of the present disclosure could be prepared, but beyond these ranges, the hydrogels could not be formed or the properties of the hydrogels were degraded.

### Example 3

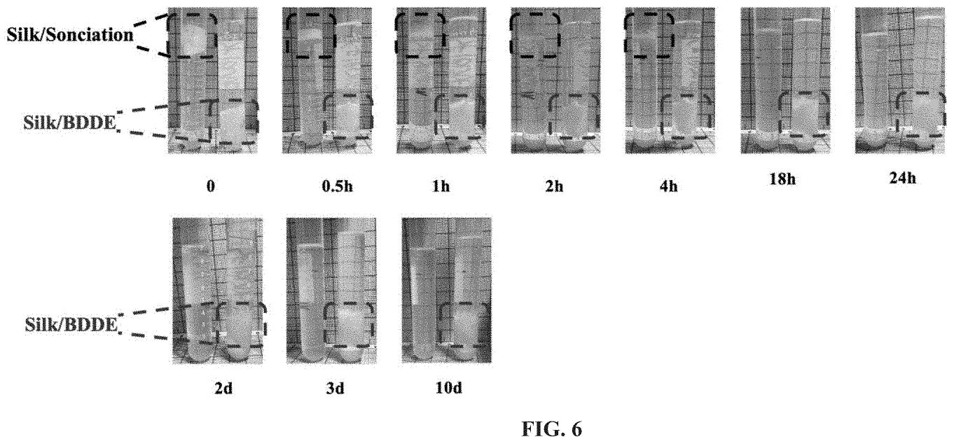

The chemically cross-linked silk hydrogel (the concentration of lithium bromide in the reaction system was 9.3 M, the concentration of silk was 200 mg/mL, the reaction was carried out at 60° C. for 1.5 h, and the silk/BDDE ratio was 1 g:200 μL) was added to the 9.3 M lithium bromide solution, and a sonication-induced physically cross-linked hydrogel was used as a control. The integrity of hydrogel blocks was observed after 0.5 h, 1 h, 2 h, 4 h, 18 h, 24 h and 2 days, 3 days and 10 days. As shown in FIG. 6, it could be seen from the figure that the sonication-induced physically cross-linked hydrogel became smaller and smaller with time, and was completely dissolved after 4 h for the reason that the mechanism of lithium bromide for dissolving silk was to destroy the β-sheets (hydrogen bonds). However, the sonication-induced physically cross-linked structure is mainly stabilized by β-sheets (hydrogen bonds) and the silk chemically cross-linked with BDDE is not affected by hydrogen bonds, so it can remain stable in lithium bromide. Its structural stability depends on the density and distribution of chemical crosslinking sites, rather than the physical effect of the hydrophobic β-sheet region on the molecular chain, so it can resist the injury and induction of the material structure by conventional silk dissolving reagents. The composition matrix has a very high stability in the conventional solution for dissolving degummed silk, and the mass loss rate does not exceed 10% after 72 h of incubation in 9.3 M lithium bromide at 60° C.

### Example 4

The silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) were respectively soaked in 80% methanol, 80% ethanol and PBS and incubated at room temperature for a period of time, and then taken out, washed with water and freeze-dried. Genipin- and HRP-catalyzed hydrogels were used as chemically cross-linked silk hydrogel controls, and sonication-induced silk hydrogels were used as physically cross-linked hydrogel controls. The secondary structure of silk was tested by FTIR infrared spectroscopy on a Nicolet 5700 Fourier transform infrared spectrometer (FTIR, Nicolet, USA) within a test wavelength range of 400 to 4000 cm⁻¹ under a liquid nitrogen freezing condition by a potassium bromide tableting method using a mortar for grinding. The amide I (1595˜1705 cm⁻¹) region on the curve was fitted with peakfit software to obtain the contents of β-sheets, α-helixes, random coils and β-turns, and the results of the content of β-sheets are listed in the table below:

| | | Treated with 80% | Soaked in | Soaked in | Soaked in |
|---|---|---|---|---|---|
| | Newly | methanol/ethanol | PBS for 7 | PBS for 15 | PBS for 30 |
| Group | prepared | for 72 h | days | days | days |
| Silk hydrogel | 23.44 ± 1.86% | 27.31 ± 2.10% | 26.23 ± 2.98% | 28.37 ± 3.22% | 29.68 ± 2.84% |
| of the present | | | | | |
| disclosure | | | | | |
| Genipin- | 27.73 ± 3.65% | 43.27 ± 2.63% | 41.88 ± 4.35% | 44.69 ± 3.47% | 47.14 ± 4.62% |
| catalyzed | | | | | |
| chemically | | | | | |
| cross-linked | | | | | |
| hydrogel | | | | | |
| HRP-catalyzed | 32.25 ± 2.36% | 39.66 ± 4.75% | 37.65 ± 5.09% | 40.23 ± 3.66% | 41.97 ± 4.03% |
| silk hydrogel | | | | | |
| of the present | | | | | |
| disclosure | | | | | |
| Sonication- | 41.16 ± 3.21% | 44.68 ± 4.56% | 44.98 ± 3.79% | 46.38 ± 4.02% | 47.66 ± 5.38% |
| induced silk | | | | | |
| hydrogel | | | | | |

It could be concluded from the table that the content of β-sheets in the secondary structure elements of the chemically cross-linked silk of the present disclosure was significantly lower than that in the sonication-induced silk (traditional physically cross-linked silk hydrogel). The stability of its secondary structure was better than that of HRP-catalyzed hydrogel under conventional physical induction with methanol, ethanol or the like, or under the condition of long-term soaking in PBS. The content of β-sheets in the secondary structure elements of the silk in the silk hydrogel was less than or equal to 32%, which can be determined by methods such as infrared spectroscopy. After incubation in PBS at 37° C. for 30 days, the content of β-sheets in the secondary structure elements of the silk was less than or equal to 40%. In the meanwhile, the inventors studied the compressive modulus (with a compressive deformation of 60%) before and after the sample incubation, and it was found that the compressive modulus of the hydrogel disclosed in the present disclosure was increased from 100 kPa to 218 kPa after incubation in ethanol, and changed to 110 kPa after incubation in PBS, with an increase of one-fold or less.

### Example 5

The silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) were cut into cylinders with a diameter of 10 mm and a height of 8 mm using a hole puncher and then evaluated by a texture analyzer (TMS-PRO, USA). The loading speed was 30 mm/min and the compressive deformation was 20%. When the hydrogel was compressed to 80% of its original height, the compressive was stopped and then the pressure was released. When a pressure sensor returned to a starting position, it was compressed again, and the compressive and release were repeated for 100 cycles. It was found that the hydrogel had good elasticity. When the deformation was 60%, the silk/BDDE hydrogel did not have any rupture, and curves after 15 compressive cycles almost overlap, indicating that the silk/BDDE hydrogel has good resilience in the compressive range of 60%. With the increase of compressive cycles, the compressive modulus of the hydrogel was increased slightly. After 15 compressive cycles, the compressive modulus was increased by only 7%, and the height recovery exceeded 95%. When the deformation was 20%, after 100 compressive cycles, the increase of compressive modulus was less than 20%, and the height recovery exceeded 80%. Since repeated compressive reduces the internal pores of the hydrogel, the compressive modulus of the hydrogel is changed, but this does not affect the good resilience of the silk/BDDE hydrogel.

### Example 6

The silk hydrogels prepared in Example 2 were freeze-dried (the concentration of lithium bromide in the reaction system was 9.3 M, the concentration of silk was 200 mg/mL, the reaction was carried out at 60° C. for 1.5 h, and the silk/BDDE ratio was 1 g:200 μL). First, the weight of a 2 mL empty centrifuge tube was measured and denoted W₀. 50 mg of freeze-dried samples were respectively weighed and suspended in 10 mM/L PBS buffer (pH 7.4) and a 5 units/mL protease solution (10 mM/L, pH 7.4). The suspensions were then sealed in centrifuge tubes (1 mL/centrifuge tube, the sample weight was denoted W₁), with 4 parallel samples in each group, and then shaken on a shaker in a 37° C. oven. The degradation solution was changed every other day, the samples were taken out within a specified time (1, 3, 5, 9, 17, 30 days), centrifuged and washed with deionized water three times, then dried at 60° C. to constant weight. The weight of the product was measured with a fine balance and denoted W₂. The mass remain of the sample was calculated as follows.

Residual mass (%)=(50−W₂)/50*100

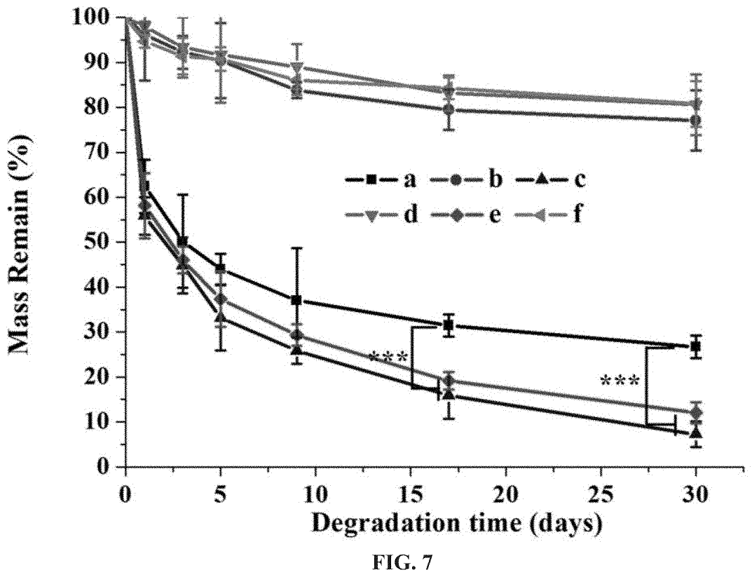

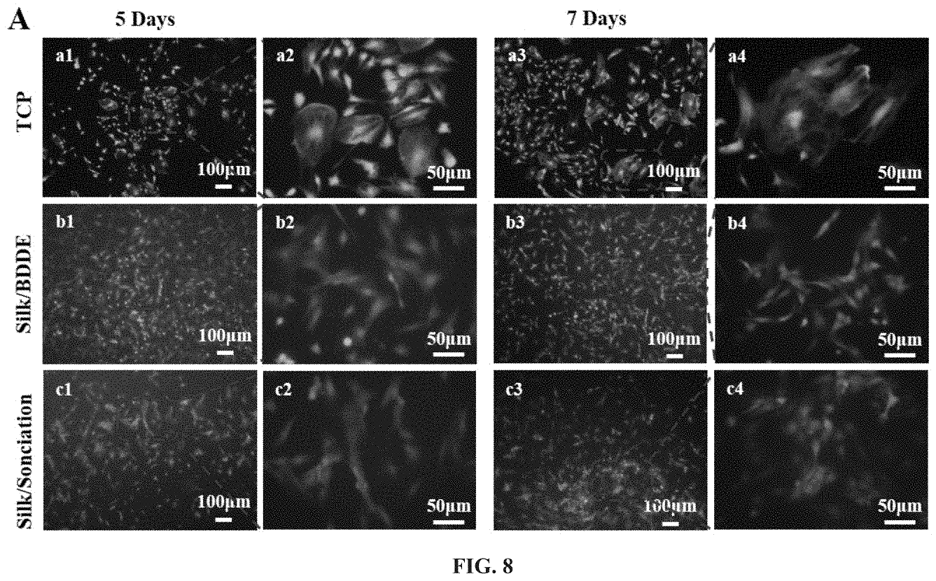

The sonication-induced physically cross-linked hydrogels and the HRP-catalyzed chemically cross-linked hydrogels were used as controls. As shown in FIG. 7, it could be concluded from the figure that the enzymatic hydrolysis resistance of the BDDE-cross-linked silk hydrogel was better than that of enzyme-catalyzed chemically cross-linked and sonication-induced physically cross-linked hydrogels. After 17 days and 30 days of enzymatic degradation, the enzymatic hydrolysis resistance of the BDDE-cross-linked silk hydrogels was significantly different from that of the enzyme-catalyzed chemically cross-linked and sonication-induced physically cross-linked hydrogels. The enzymatic hydrolysis resistance of the enzyme-catalyzed chemically cross-linked hydrogels was better than that of the sonication-induced physically cross-linked hydrogels, but without significant difference. This will be of great significance to the research and application in the direction of medical and cosmetic filling, and will effectively improve the effective filling time.

### Example 7

The lithium bromide silk-dissolving solution added with a crosslinking agent (in Example 2, the concentration of lithium bromide in the reaction system was 9.3 M, the concentration of silk was 200 mg/mL, and the silk/BDDE ratio was 1 g:200 μL) and the ungelatinized silk solution after sonication treatment were injected into a 24-well plate, 200 μL per well, and 6 parallel samples were taken from each group. After crosslinking, the silk/BDDE hydrogel was washed with dialysate, air-dried on an ultra-clean bench for 2 h, and sterilized by irradiation at a dose of 25 kGy.

Mesenchymal stem cells (rBMSCs) of SD rats were cultured to the third passage, and then digested with trypsin and pipetted to form a suspension. The suspension was seeded into the 24-well plate at a dose of 1×10⁴ cells/mL, 1 mL per well. The 24-well plate was then place in a 37° C. cell incubator with 5% CO₂ for incubation. The control group was denoted TCP. On the 1st, 3rd, 5th, 7th, and 10th days, the medium of the 24-well plate was removed, 1 mL of fresh DMEM containing 10% of Alamar Blue was added, and the cells were then incubated for 2.5 h in the incubator. 70 μL of the culture solution was then pipetted to a black 96-well plate, and 6 parallel samples were taken from each group. The fluorescence values were detected with a microplate reader at the excitation wavelength of 560 nm and the emission wavelength of 590 nm.

Live cell staining (calcein): After cells were incubated in samples in the 24-well plate for 5 days and 7 days, respectively, the medium in the well plate was removed and discarded, and then the samples were separately stained for live cells. 0.5 μL of calcein (622.55 Da, Gibco, USA) was well mixed with 1 mL of medium to serve as a staining solution and 1 mL of the staining solution was then added to the samples. All these operations were carried out in the dark. Next, the samples were incubated in a 37° C. cell incubator for 35 mM, the staining solution was then discarded, the samples were washed twice with 10 mM of sterile PBS solution, and the PBS solution was then discarded. The incubated cells were then observed under an inverted fluorescence microscope for their growth morphology.

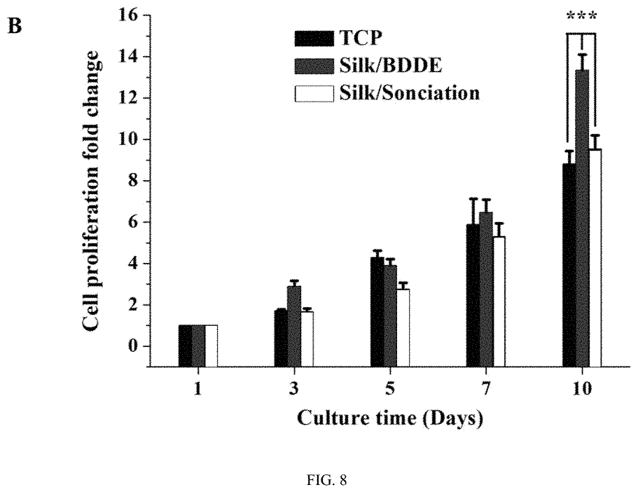

The sonication-induced physically cross-linked hydrogels were used as controls. As shown in FIG. 8, it could be concluded from the figure that RMSCs proliferate continuously on the surfaces of BDDE-cross-linked hydrogels, the sonication-induced physically cross-linked hydrogels and TCP, and the proliferation rate of cells on the surface of BDDE-cross-linked hydrogels was higher than that of sonication-induced physically cross-linked hydrogels, with a significant difference on day 3 and day 5.

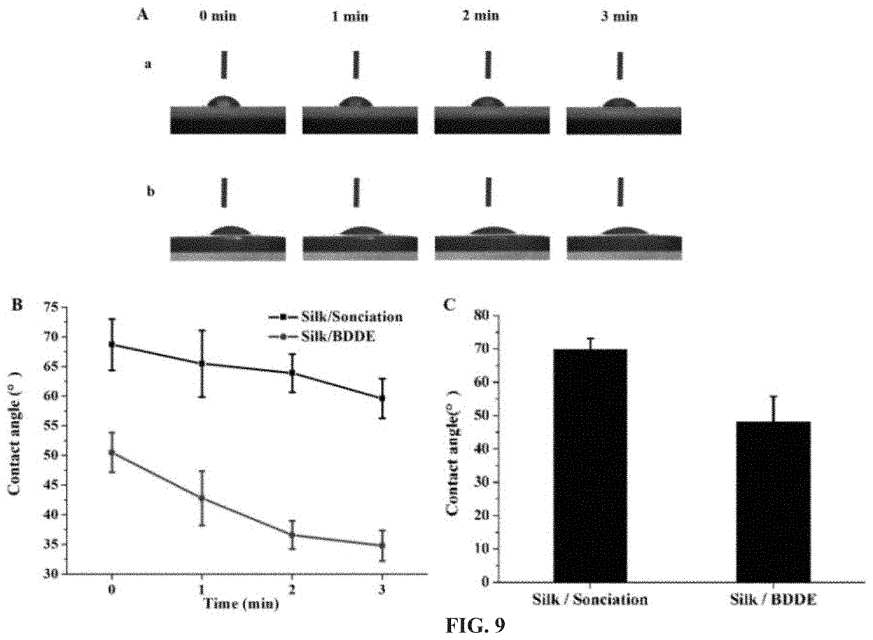

The inventors further studied the surface hydrophilicity and hydrophobicity of the BDDE-cross-linked hydrogel. As shown in FIG. 9, it was found that the static contact angle of the surface of the BDDE-cross-linked hydrogel was 48.2±7.6°, which was lower than that of the sonication-induced physically cross-linked hydrogel (69.9±3.3°). It has been widely reported that the surface hydrophilicity of a material is beneficial to cell adhesion and proliferation, indicating that the BDDE-cross-linked hydrogels not only have good biosafety, but also can promote cell proliferation as compared with the physically cross-linked hydrogels, which is very important for the application of the hydrogels in the field of biomedical materials.

### Example 8

Nine SPF SD rats weighing 60-150 g were selected for subcutaneous embedding. Samples (the silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL)) were sterilized in advance by irradiation, and the size of the samples was 0.5×0.5 cm. Before subcutaneous embedding, the rats were anesthetized with a dose of chloral hydrate corresponding to the body weight of the rats, and then the backs of the rats were shaved. An incision of 1.5-1.8 cm was made in the epithelium of the back of each rat, and the sample was embedded in the skin and sutured. The rats were then disinfected and sterilized with iodophor and then put back into cages and raised. At the indicated time intervals (3, 7, 28 days), 3 rats were taken out and sacrificed with an overdose of chloral hydrate. The samples in the back were taken out and fixed in formalin for histological analysis.

H&E staining: sample sections were first soaked in xylene for 20 min and the xylene was then discarded. Next, the sample sections were soaked again in new xylene for 20 min and the xylene was then discarded. The sample sections were further soaked in anhydrous ethanol for 5 min, the anhydrous ethanol was then discarded, and the sample sections were then soaked again in new anhydrous ethanol for 5 min, and then the anhydrous ethanol was discarded. The sample sections were further soaked in 75% ethanol for 5 min and washed with pure water. The sections were stained with hematoxylin staining solution for 3 to 5 min, then washed with pure water, then put into a differentiation solution, then washed with pure water, and put into a blue-returning solution, and then rinsed with pure water. The sections were dehydrated with 85% and 95% alcohol respectively, 5 min each time, and then stained in an eosin staining solution for 5 min. The sections were then soaked in absolute ethanol for 5 min and the anhydrous ethanol was then discarded, and these operations were repeated three times. The sample sections were soaked in xylene for 5 min, the xylene was then discharged, the sample sections were soaked again in new xylene for 5 min and the xylene was then discharged. The sections were then sealed with neutral gum, and observed under an inverted fluorescence microscope (Axio Vert A1, Germany) and the images of the sections were acquired.

Masson staining the sample sections were first soaked in xylene for 20 min, the xylene was then discarded. Next, the sample sections were soaked again in new xylene for 20 min and the xylene was then discarded. The sample sections were further soaked in anhydrous ethanol for 5 min, the anhydrous ethanol was then discarded, and the sample sections were then soaked again in new anhydrous ethanol for 5 min, and then the anhydrous ethanol was discarded. The sample sections were further soaked in 75% ethanol for 5 min and washed with pure water. The sections were then soaked in Masson A solution overnight and then washed with pure water. Mix Masson B solution and Masson C solution were well mixed in equal proportions, and the sections were then soaked in the mixed staining solution for 1 min, and then wash with pure water. 1 mL of hydrochloric acid was diluted to 100 mL with absolute ethanol, and the sections were put in the resulting solution for differentiation, and then washed with pure water. The sections were further soaked in Masson D solution for 6 min, then washed with pure water, and then soaked in Masson E solution for 1 min, the Masson E solution was then drained and the sections were further soaked in Masson F solution for 2 s to 30 s. The sections were then taking out, washed with 1% glacial acetic acid, and then dehydrated with absolute ethanol. After dehydration, the sections were soaked in new absolute ethanol for 5 min and the absolute ethanol was then discarded. The sections were further soaked in xylene for 5 min, and then taken out and sealed with neutral gum. The sections were observed under an inverted fluorescence microscope (Axio Vert A1, Germany), and their images were acquired. The collagen deposition was analyzed by Image J software.

Immunofluorescence staining: the sample sections were first soaked in xylene for 15 min, the xylene was then discarded. Next, the sample sections were soaked again in new xylene for 15 min and the xylene was then discarded. The sample sections were further soaked in anhydrous ethanol for 5 min, the anhydrous ethanol was then discarded, and the sample sections were then soaked again in new anhydrous ethanol for 5 min, and then the anhydrous ethanol was discarded. The sample sections were further soaked in 75% ethanol for 5 min and washed with pure water. The tissue sections were saturated with an EDTA antigen retrieval buffer (pH=8.0) and then put in a repair box and subjected to antigen retrieval in a microwave oven. The tissue sections were first heated to boiling on medium heat, heating was then stopped, and then the tissue sections were slowly heated on medium-low heat for 7 min. The tissue sections were then taken out and cooled at room temperature, glass slides were soaked in PBS and washed on a decolorizing shaker for 5 min, and these operations were repeated three times. The sections were taken out and dried slightly, circles were drawn around the tissue with histochemical strokes, and the PBS on the glass slides was then shaken off. 3% BSA was added dropwise, and the sections were sealed for 30 min. The sections were taken out, the liquid on the slides was gently shaken off, a primary antibody was added dropwise, and then the cells were incubated overnight at 4° C. in a humidified box with a small amount of water. The slides were then soaked in PBS and washed on a decolorizing shaker for 5 min, and the soaking and washing operations were carried out three times. The slides were then taken out and dried slightly. A secondary antibody was dropwise added into the circle to cover the tissue, and the sections were set still in the dark for 50 min. The slides were soaked in PBS again and washed on the decolorizing shaker for 5 min, and the soaking and washing operations were carried out three times. The slides were then taken out and dried slightly. A DAPI staining solution was dropwise added into the circle and the sections were set still at room temperature in the dark for 10 min. The slides were soaked again in PBS and washed on the decolorizing shaker for 5 min, and the soaking and washing operations were carried out three times. The slides were then taken out. An autofluorescence quencher was dropped into the circle, and after 5 min, the slides were rinsed with pure water for 10 min. After the sections were dried slightly, anti-fluorescence quenching mounting medium was added dropwise for sealing the sections. The sections were observed under laser confocal, (DAPI has a UV excitation wavelength of 330 to 380 nm and an emission wavelength of 420 nm and emits blue light; FITC has an excitation wavelength of 465 to 495 nm and an emission wavelength of 515 to 555 nm and emits green light; CY 3 has an excitation wavelength of 510 to 560 nm and an emission wavelength of 590 nm and emits red light) and their images were acquired and analyzed by Image J software.

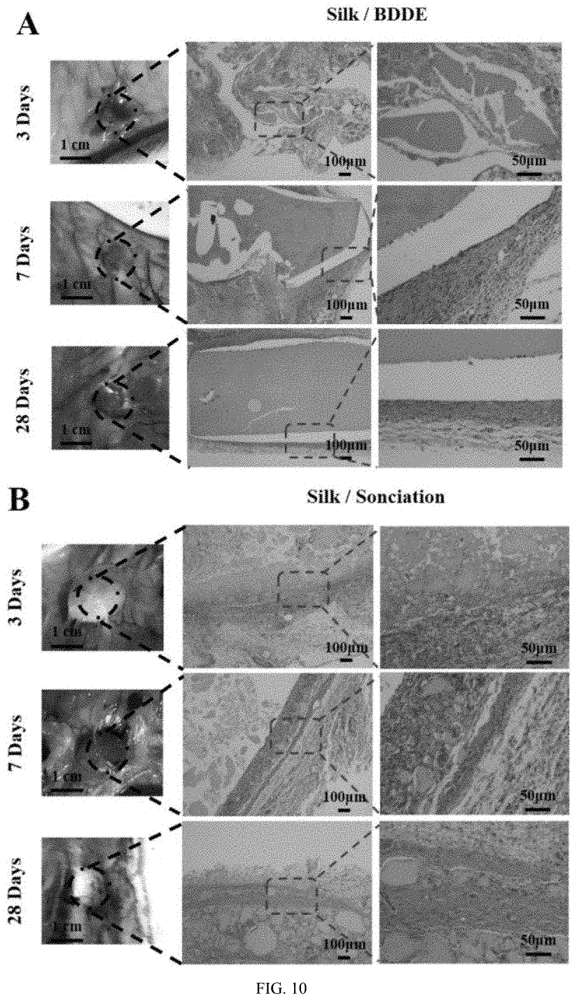

The sonication-induced physically cross-linked hydrogels were used as controls. As shown in FIG. 10, it could be concluded from the figure that the interface between the BDDE-cross-linked hydrogel and the surrounding tissue was clear, almost without envelope formed. However, sonication-induced physical cross-linked hydrogels were broken into small pieces, which accelerated the migration of surrounding cells. An about 200 μm film was formed in 7 days, and cells almost migrated to gaps between the hydrogel blocks in 28 days.

### Example 9

A. The silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) were ground in a tissue grinder and then poured out.

B. According to the hydrogel crosslinking reaction conditions screened in Example 2 that the concentration of silk was 200 mg/mL, the silk/BDDE ratio was 1 g:200 μL, and the concentration of lithium bromide in the reaction system was 9.3 M, a mixed solution of the silk silk-dissolving solution and BDDE was prepared and immediately added dropwise as an aqueous phase to an oil phase that had been preheated to 60° C. and was in motion.

(1) The rotation speed of the oil phase was fixed at 500 rpm, and the oil-water ratio was changed to 1:1, 2:1, 5:1, 10:1, 100:1, and 500:1. After the oil phase and the water phase were well mixed and set in an oven with a temperature of 60° C. to react for 3 h. The reaction system was observed to check whether a spherical hydrogel was formed therein. The results are as follows:

| Oil-water ratio (v/v) | Hydrogel form |
|---|---|
| 1:1 | Bulk hydrogel |
| 2:1 | Bulk hydrogel |
| 5:1 | Spherical hydrogel |
| 10:1 | Spherical hydrogel |
| 100:1 | Spherical hydrogel |
| 500:1 | Spherical hydrogel |

It could be seen from the results in the table that when the oil-water ratio was 1:1 and 2:1, because the volume of the water phase was too large, the distance between the round hydrogel spheres formed in the moving oil phase was too small and the spheres were easily aggregated. Aggregates of hydrogel spheres those were not fully cross-linked gradually crosslink to form bulk hydrogels, instead of cross-linked gel spheres. When the oil-water ratio was 5:1, 10:1, 100:1, and 500:1, hydrogel spheres could be formed. Considering the granulation efficiency, the inventors believe that the oil-water ratio of 10:1 is the best reaction condition.

(2) The oil-water ratio was fixed at 10:1, and the rotation speed of the oil phase was changed to 50 rpm for stirring, 100 rpm for stirring, 500 rpm for stirring, 1000 rpm for stirring, 5000 rpm for homogenating, and 10000 rpm for homogenating. After the oil phase and the water phase were well mixed and set in an oven with a temperature of 60° C. to react for 3 h. The reaction system was observed to check whether hydrogel spheres were formed therein. The results are shown in the table below.

| | |
|---|---|
| Rotation speed | Hydrogel form |

| | | |
|---|---|---|
| 50 | rpm | Bulk hydrogel |
| 100 | rpm | Spherical hydrogel |
| 1000 | rpm | Spherical hydrogel |
| 5000 | rpm | Spherical hydrogel |
| 10000 | rpm | Spherical hydrogel |

It could be seen from the results in the table that when the rotation speed was 50 rpm, because the oil phase moved slowly, the motion amplitude and frequency of the hydrogel spheres in the oil phase were too small, and the spheres were easily collided and aggregated. Aggregates of hydrogel spheres those were not fully cross-linked gradually crosslink to form bulk hydrogels, instead of cross-linked hydrogel spheres. When the rotation speed of the oil phase was 100 rpm, 500 rpm, 1000 rpm, 5000 rpm and 10000 rpm, hydrogel spheres could be formed.

The above-mentioned hydrogel spheres were filtered out from the oil phase, and oil on the surfaces of the spheres was first washed away with an organic solvent, and then washed with water. The collected hydrogel spheres were filtered through sieves with different mesh numbers, and the hydrogel spheres with sizes of 300 μm or above, 100 μm to 300 μm, and 100 μm or below were collected, freeze-dried and weighed. The yield was calculated.

Yield of hydrogel spheres=W₂/W₁*100%

W₁ represents the total weight of hydrogel spheres under all mesh numbers after freeze-drying, and W₂ represents the weight of hydrogel spheres within the ranges of the calculated mesh numbers after freeze-drying. The results are shown in the table below.

| | 300 μm or above | 100-300 μm | 100 μm or below |
|---|---|---|---|
| 100 rpm for | 79.12% ± 2.42% | 18.95% ± 0.78% | 2.11% ± 0.87% |
| stirring | | | |
| 1000 rpm for | 39.34% ± 3.25% | 52.41% ± 2.21% | 8.22% ± 5.10% |
| stirring | | | |
| 5000 rpm for | 0 | 8.82 ± 2.31% | 92.21% ± 2.06% |
| homogenating | | | |
| 10000 rpm for | 0 | 3.31 ± 1.35% | 98.11% ± 1.12% |
| homogenating | | | |

It could be seen from the data in the table that in the hydrogel spheres produced under the stirring condition of 100 rpm, most had sizes of 300 μm or above, with a yield of 79.12%±2.42%, and a minority had sizes of 100 μm to 300 μm and 100 μm or below; in the hydrogel spheres produced under the stirring condition of 1000 rpm, the yield of hydrogel spheres having sizes between 100 μm and 300 μm reached highest and was 52.41%±2.21%, followed by hydrogel spheres having sizes of 300 μm or below with the yield of 39.34%±3.25%, the least being hydrogel spheres having sizes of 100 μm or below; in the hydrogel spheres produced under the homogenating conditions of 5000 rpm and 10000 rpm, most had sizes of 100 μm or below, with the yield of 92.21%±2.06% and 98.11%±1.12%, respectively. In the range of 100 rpm to 10000 rpm, ideal silk hydrogel microspheres can be obtained. In the range of the oil-water ratio being greater than 1:1, ideal silk hydrogel microspheres can be obtained. In the range of the oil-water ratio being greater than 2:1 to 500:1, silk hydrogel microspheres with more excellent performance can be obtained.

### Example 10

The hydrogel particles A and hydrogel spheres B prepared by the method in Example 9 were filtered with sieves of different mesh numbers, and the hydrogel spheres with sizes of 300 μm or above, 100 μm to 300 μm, and 100 μm or below were collected respectively, and water on the surfaces of the hydrogel spheres was absorbed as much as possible with filter paper. The content of silk in the hydrogel spheres was determined by a drying weighing method. The hydrogel spheres were put into a 1 mL syringe and then injected at a speed of 4 mL/min through a 27 G needle, and a push-pull force meter was used to record the peak value and mean value of the injection force during the injection. If the injection could not be down, no injection force value was recorded. The results are as follows:

| | 300 μm or above | 100-300 μm | 100 μm or below |
|---|---|---|---|
| Peak injection | N.J. | N.J. | 18.23 ± 3.44 |
| force (N) | | | |
| Mean injection | N.J. | N.J. | 15.34 ± 3.21 |
| force (N) | | | |

Note:
N.J. means that it cannot be injected under the condition.

It could be seen from the table that hydrogel spheres with a size of 300 μm or above and 100-300 μm could not be directly injected through a 27 G needle. Hydrogel spheres with a size of 100 μm or below could be injected through a 27 G needle, but the peak injection force and the mean injection force were both greater than 10 N.

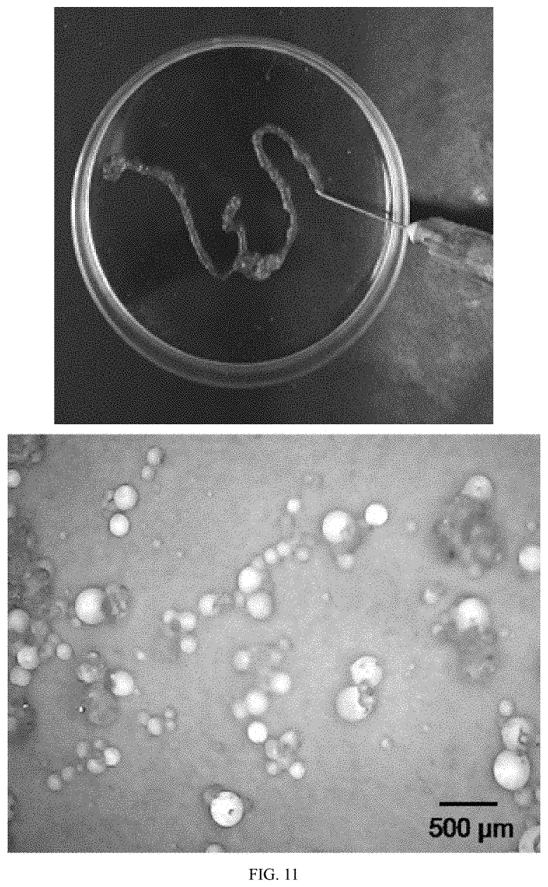

A small amount of lubricant, such as sodium hyaluronate, carboxymethyl cellulose, hydroxyethyl cellulose, and the like, was added to the hydrogel spheres with a size of 100 μm to 300 μm to obtain a chemically cross-linked silk hydrogel sphere composition. The macroscopic and microscopic morphologies of the composition were shown in FIG. 11. In the composition, the fraction (v/v) of hydrogel spheres to the total volume of the composition, the fraction of silk to the total mass of the composition (w/w), the peak injection force and the mean injection force are shown in the table below:

| Fraction of hydrogel | | | |
|---|---|---|---|
| spheres to the total | Fraction of | | |
| volume of the | silk to the | Peak injection | Mean injection |
| composition (v/v) | total mass (w/w) | force | force |
| 50% | 5% | 7.32 ± 2.71 | 6.43 ± 1.37 |
| 70% | 10% | 10.11 ± 1.32 | 9.56 ± 0.48 |
| 90% | 15% | 9.87 ± 1.01 | 8.76 ± 0.92 |
| 100% | 20% | N.J. | N.J. |

Note:
N.J. means that it cannot be injected under the condition.

As disclosed in Chinese patent CN 102836465 B, silk fibroin was set at room temperature to form a hydrogel, the hydrogel was mechanically crushed to obtain micro particles, and then the silk micro particles were added to a hyaluronic acid solution, and a crosslinking agent was added for crosslinking to obtain an injectable fibroin-hyaluronic acid composite hydrogel. The mass ratio of silk to hyaluronic acid in this composite hydrogel was at most 1:10, that is, the content of silk did not exceed 10%. The formation of the hydrogel by naturally placing the silk at room temperature refers to the formation of β-sheets in the silk, which belongs to physical crosslinking. A physically cross-linked silk hydrogel is hard and has poor elasticity. When pushed, the physically cross-linked silk hydrogel has almost no space for compressive, and it is difficult to produce sliding. It requires a large amount of viscous medium to drive the injection of the hydrogel. The BDDE-cross-linked fibroin hydrogel has good elasticity and certain lubricity, and can be injected through a fine needle just by the lubricity of its surface and its good elasticity when pushed.

### Example 11

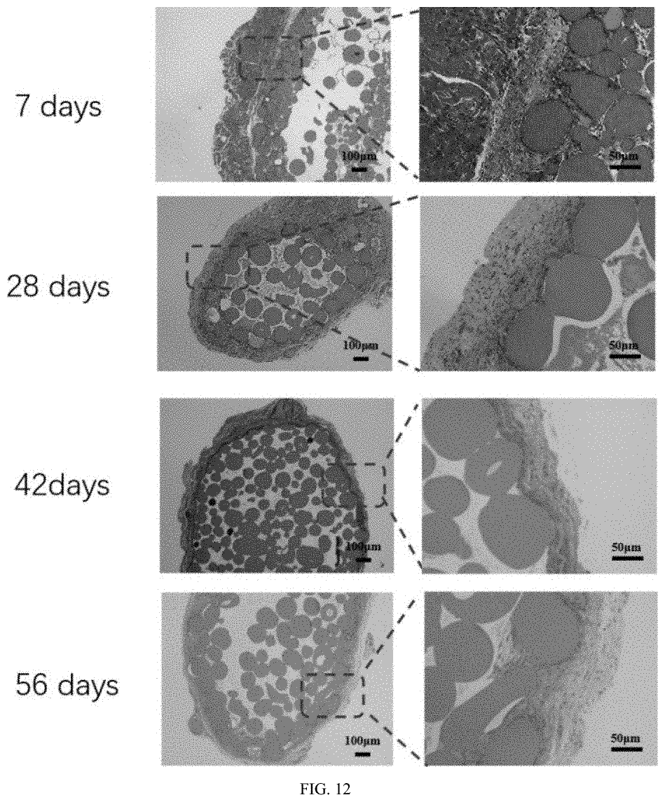

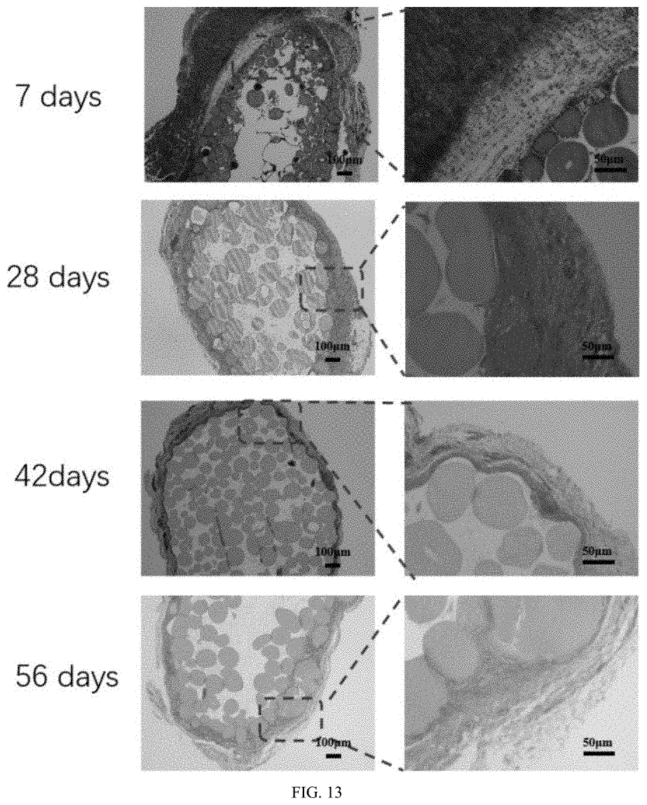

The composition of chemically cross-linked silk hydrogel spheres (the gel spheres accounted for 90% of the total volume of the composition, the silk accounted for 15% of the total mass of the composition, the particle size was within a range of 100 μm to 300 μm, and 1% (w/v) hyaluronic acid was added as a lubricant) prepared by the method of Example 10 was injected into the subcutaneous tissue on the back of the rats through a 27 G needle, in a dose of 1 mL at each point. On day 7, day 28, day 42, and day 56, the rats were sacrificed, and the samples and surrounding tissues were taken out, fixed with formalin and then with paraffin, then sectioned, subjected to H&E staining (FIG. 12) and MASSON staining (FIG. 13), and observed under microscope. It could be seen from the figures that the interface between the injectable silk hydrogel spheres and the surrounding tissue was clear, almost without envelope formed, that is, the silk hydrogel spheres almost had no inflammatory response after 8 weeks of subcutaneous use in rats.

### Example 12

The reduction of synovial fluid and the change of its composition will lead to change in the internal environment of the knee joint and aggravate the wear of the articular cartilage surface. When joint pain occurs, injecting highly elastic silk hydrogel spheres into the joint can change the internal environment of the joint, restore the synovial fluid under pathological conditions to the normal state, and achieve the purpose of lubricating the knee joint.

The composition of chemically cross-linked silk hydrogel spheres (the hydrogel spheres accounted for 90% of the total volume and the silk accounted for 15% of the total mass, the particle size was within a range of 100 μm to 300 μm, and 1% hyaluronic acid was added as a lubricant) prepared by the method of Example 10 was injected into the joint cavities of rats. 24 SD rats (250 g/rat) were randomly divided into a model group and a treatment group, with 12 rats in each group. An osteoarthritis model was established in each rat by ACLT modeling method (unilateral modeling). At the third week after the establishment of OA model, the groups were intervened respectively. The model group was intervened with physiological saline and the treatment group was intervened with the chemically cross-linked silk hydrogel spheres, with an injection volume of 50 μL. After the establishment of OA model, an electronic pain threshold detector was used to measure the electronic pain thresholds of the knee joints of rats to reflect the pain symptoms caused by osteoarthritis. Specifically, the left and right feet of the rat were pressed on a test bench. When the rat was struggling, the electronic pain threshold detector automatically sensed to stop and count. The lower the tenderness threshold, the more severe the disease. Measurements were made once a week after the establishment of OA model was completed, and statistics and difference comparison were carried out.

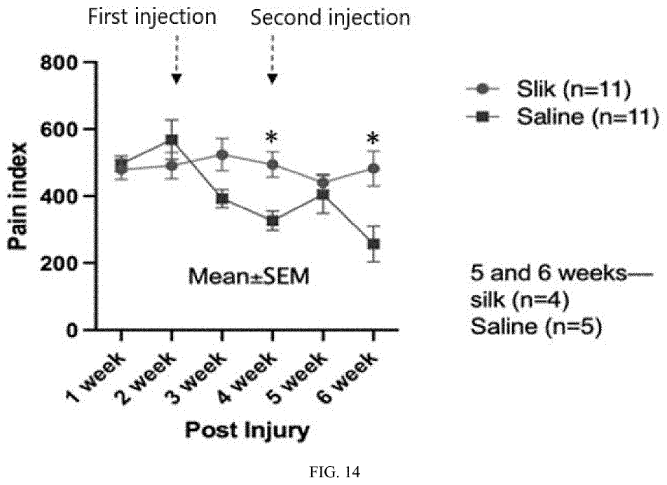

The test results were shown in FIG. 14. Through result analysis, it could be found that one week and two weeks after the establishment of OA model, there was no significant difference in the pain threshold between the model group and the treatment group. First administration was performed at the second week after the establishment of OA model, and the pain threshold of rats in the model group was obviously lower than that of the treatment group in the third and fourth weeks, and there was a significant difference in the pain threshold between the rats in the model group and the rats in the treatment group at the fourth week. This indicates that the injection of silk hydrogel spheres can ease the pain of rats caused by arthritis. Second administration was performed after the pain threshold was tested at the fourth week. By the fifth and sixth weeks, the pain threshold of the rats in the treatment group was still higher than that of the rats in the model group, and there was a significant difference in the pain threshold between the model group and the treatment group at the sixth week. This may be because the injected highly elastic silk hydrogel spheres have a certain lubricating effect on the joints and improve the viscosity and elasticity of the joint fluid.

### Example 13

Dry eye is a multifactorial disease of tears and the surface of the eyeball, which may cause discomfort, visual disturbance and tear film instability. The epithelial cells on the ocular surface of normal organisms are cup-shaped, and their function is to secrete enough mucin to keep the ocular surface moist; however, epithelial cells on the ocular surface of patients with dry eye cannot take this function. To reduce pain, the use of highly elastic silk hydrogel ball eye drops can lubricate the surface of the cornea and relieve eye discomfort.

The composition of chemically cross-linked silk hydrogel spheres (the hydrogel spheres accounted for 90% of the total volume and the silk accounted for 15% of the total mass, and the particle size was within a range of 50 μm to 100 μm) prepared by the method of Example 10 was applied to postoperative treatment of dry eye model rabbits. Twelve healthy New Zealand white rabbits, half male and half female, without any medical history, weighed 2.0 to 2.5 kg, had no eye disease and abnormality before the experiment, and were pre-fed for 7 days. After the animals were fixed, general anesthesia was performed through the ear vein, and a proparacaine hydrochloride eye drop (applied 3 drops to the eye) was used for local anesthesia. After that, the animals were placed on the test bench without any operation on the right eye. The left lacrimal gland, harderian gland and third eyelid were removed to establish a rabbit model of dry eye with insufficient tear production. On day 14 after the operation, the animals were randomly divided into 2 groups: silk group and control group, with 6 animals in each group. The control group did not receive any treatment, while animals in the silk group were treated with a silk hydrogel sphere eye drop 3 times a day, 2 droplets each time. The animals were administrated for 28 days. The Schirmer I test was performed before administration and 7 days, 14 days, 21 days, and 28 days after administration to evaluate tear secretion. The Schirmer I test was carried out in an indoor environment with moderate humidity and brightness. A piece of 5×35 mm filter paper was placed at ⅓ of the junction of the middle and outer parts of the inferior conjunctival sac. 5 min later, the filter paper was taken out and the length of the wet part of the filter paper was read according to scales and recorded. The mean value of three consecutive measurements was taken as the result of the Schirmer I test. Normally, the wet length of the filter paper is greater than 10 mm/5 min.

As shown in the table below, there was no difference in the tear secretion between the control group and the silk group before administration; 7 days after administration, the tear secretion of the silk group was higher than that of the control group (P<0.05); 14 days after administration, the tear secretion of the silk group was significantly improved relative to that of the control group (P<0.01); and 21 days and 28 days after administration, there was obvious difference in the tear secretion between the silk group and the control group. This indicates that silk hydrogel sphere eye drop improves the dry eye.

Changes of tear secretion of animals before and after administration
at different times (mean ± SD, n = 6)

| | Schirmer I value (mm/5 min) | | | | |
|---|---|---|---|---|---|
| Group | 0 days | 7 days | 14 days | 21 days | 28 days |
| Silk | 4.33 ± 0.14 | 9.82 ± 0.54* | 11.74 ± 1.28** | 15.87 ± 0.82** | 18.95 ± 1.78** |
| group | | | | | |
| Control | 4.25 ± 0.23 | 7.06 ± 0.79 | 7.43 ± 0.86 | 8.86 ± 0.75 | 10.31 ± 1.01 |
| group | | | | | |

Note:
Compared with the control group,
*P < 0.05,
**P < 0.01.

### Example 14

Age-related macular degeneration (AMD) is an eye disease characterized by loss of central vision and is the leading cause of severe and irreversible loss of vision. Currently, anti-VEGF drugs have become the standard treatment for AMD. Silk hydrogels are used as injectable formulation carriers for the continuous delivery of anti-VEGF drugs (such as bevacizumab), so that the daily release rates of the anti-VEGF drugs (such as bevacizumab) are maintained within their therapeutic range, thereby reducing administration frequency and improving the comfort of patients.

The composition of chemically cross-linked silk hydrogel spheres (the hydrogel spheres accounted for 90% of the total volume and the silk accounted for 15% of the total mass, and the particle size was within a range of 50 μm to 100 μm) prepared by the method of Example 10 was dried in a 60° C. blast drying oven overnight. The dried silk hydrogel spheres were placed in a bevacizumab solution to swell and then taken out after the volume of the silk hydrogel spheres did not increase any longer. The bevacizumab on the surface was washed off with pure water, thus obtaining bevacizumab-loaded silk hydrogel spheres.

Twelve healthy New Zealand white rabbits, male or female, weighed 3.0 kg to 3.5 kg, and underwent eye examination to confirm that they had no disease in the anterior and posterior segments. The rabbits were randomly divided into 2 groups: silk group and control group, with 6 rabbits in each group. The experimental animals were anesthetized with 35 mg/kg ketamine hydrochloride combined with 5 mg/kg xylazine hydrochloride through intramuscular injection, and then fixed on an operating table. One droplet of a 0.4% oxybuprocaine hydrochloride eye drop was instilled into the conjunctival sac, and the conjunctival sac was washed with 0.9% normal saline for 3 min. A piece of sterile towel was spread, and the eyelid was opened with an eyelid opener. A puncture knife was first used to pierce the cornea about ⅔ of the thickness under the operating microscope and then a 1 mL syringe was inserted into the anterior chamber to remove about 0.05 mL of aqueous humor. After the needle was pulled out, the puncture opening was pressed immediately with a sterile cotton swab, and another 1 mL syringe was used to inject 0.05 mL of bevacizumab-loaded silk hydrogel spheres (the load of bevacizumab was 1.25 mg/mL) or 0.05 mL of normal saline. After the injection, the needle was pulled quickly and the injection hole was pressed with a cotton swab for a while to prevent the drug from flowing out. After the injection, a tarivid eye drop was instilled in the eyes 4 times a day. Examination with a slit-lamp microscope was performed every day within one week after drug injection, and direct fundus examination with an ophthalmoscope was performed after mydriasis. One week later, examination with a slit-lamp microscope and direct fundus examination with an ophthalmoscope were performed once a week. The results at different time points after injection of bevacizumab-loaded silk hydrogel spheres into the anterior chamber showed that no inflammatory response of anterior segment tissue, corneal edema, lens opacity, vitreous opacity, retinal edema, bleeding, exudation or and other phenomena were observed under the slit-lamp microscope and the ophthalmoscope, and this indicates that the injection of bevacizumab-loaded silk hydrogel spheres have no obvious toxic and side effects on the anterior segment of rabbits during the observation period, which provides reliable evidence for clinically safe application of silk hydrogel sphere injection into the anterior chamber.

### Example 15

The silk hydrogel prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) was taken and evenly spread a thin layer on the bottom of a shallow flat-bottomed container to obtain a BDDE-cross-linked silk film. The BDDE-cross-linked silk film has high transparency and is soft and has a certain degree of elasticity after being wetted with water, thus being an ideal material for contact lenses.

### Example 16

Bone tissue, as one of the most important tissues and organs in the human body, undertakes many important functions. Bone tissue can repair itself. However, when a major injury occurs, bone tissue cannot reach an ideal state through self-repair. Medical personnel implement bone repair mainly by autologous transplantation, allotransplantation and bone tissue engineering means. As a bone repair biomaterial for bone tissue engineering, it needs to have good biocompatibility, biodegradability matching the growth of bone tissue, certain osteoinductive and conductive properties, certain mechanical properties, and a three-dimensional, interconnected porous structure to support the adhesion, growth and proliferation of seed cells. A porous silk scaffold has a three-dimensional structure, and its interconnected porous structure can provide support for the adhesion, growth and proliferation of cells and channels for the discharge of metabolites.

The silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) were freeze-dried to obtain porous BDDE-cross-linked silk scaffolds. The control group included porous silk scaffolds obtained by freeze-drying the aqueous solutions of silk having the same concentration and then fumigating with methanol for 2 h. The porous scaffolds were tested for porosity by liquid replacement. The weight of a freeze-dried silk scaffold sample was measured on a balance and denoted W. The porous silk scaffold was soaked in hexane with a volume of V1 for 15 min, and the total solution volume of hexane at that moment was denoted V2. The sample was then taken out and the volume of the remaining hexane was denoted V3. The porosity of the porous scaffold was calculated as follows: P (%)=(V1−V3)/(V2−V3)*100%, where P represents the porosity of the porous silk scaffold. The mechanical properties of the porous silk scaffold were tested by a texture analyzer. The porous silk scaffold was cut into a cube with a side length of 10 mm, the speed of a horizontal head was set to be 10 mm/min, and the compressive displacement was set to be 5 mm. The test was repeated 5 times for each sample.

The test data of porosity and mechanical properties of porous silk scaffolds are shown in the table below.

| | | Compressive | Compressive |
|---|---|---|---|
| | Porosity (%) | strength (kPa) | modulus (kPa) |
| BDDE-cross-linked | 74.44 ± 5.31 | 77.28 ± 3.54 | 311.98 ± 43.72 |
| silk scaffold | | | |
| Methanol-fumigated | 67.54 ± 3.20 | 65.32 ± 4.21 | 233.84 ± 39.93 |
| silk scaffold | | | |

It could be seen from the table that compared with the scaffold material obtained by directly freeze-drying the aqueous solution of silk, the BDDE-cross-linked silk scaffold had higher porosity. Generally speaking, the materials with higher porosity can provide more space for cell growth as human tissue engineering scaffold materials, so higher porosity is often pursued under the premise of maintaining certain mechanical properties. It could also be seen from the table that the chemically cross-linked silk scaffold had a compressive strength of 77.28 kPa and a compressive modulus of 311.98 kPa, and the compressive strength and compressive modulus of the methanol-fumigated silk scaffold were 65.32 kPa and 233.84 kPa, respectively, indicating that the BDDE-cross-linked silk scaffold has better mechanical properties.

### Example 17

For the treatment of fractures, it has gone through the process from strong fixation to biological fixation. The traditional bone screw and bone plate systems have problems such as infection, stress shielding, displacement of the nail plate, and subjective pain or paresthesia. With the development of absorbable polymer materials, such as the application of polyacetic acid, polyglycolic acid, and polyacetic acid glycolic acid for absorbable bone plates and screws, the risk of secondary surgery can be reduced, and the stress can be gradually transferred onto healed bones, thereby promoting bone regeneration. The use of fixation materials with low elastic modulus and good biocompatibility is a new challenge for intraosseous implants due to the advancement of fracture treatment concepts.

The silk hydrogels prepared in Example 2 (the concentration of lithium bromide in the reaction system was 9.3 M, the concentration of silk was 200 mg/mL, the reaction was carried out at 60° C. for 1.5 h, and the silk/BDDE ratio was 1 g:200 μL) were dried slowly in a 4° C. blast drying oven and then polished and machined into cylindrical rods to obtain BDDE-cross-linked silk hard bone materials.

The biomechanical properties of the BDDE-cross-linked silk hard bone materials were tested by a biomechanical tester. The cylindrical silk rods used for the test were 17.5 cm in length, 0.45 cm in diameter, and the span of three-point bending test was 5 mm After a sample was installed on one end of a fixture (a force sensor), the force sensor returned to zero; the other end of the sample was then clamped. The sample was tightened with a certain pre-tightening force. The voltage control mode was selected to pull the sample. Test parameters were set, the test was then carried out and data were saved. The test results are shown in the table below.

Biomechanical properties of the BDDE-cross-linked silk hard bone material

| Flexural | | Elastic modulus | Elastic modulus |
|---|---|---|---|
| strength | Elastic modulus | of cortical | of stainless |
| (MPa) | (GPa) | bone (GPa) | steel (GPa) |
| 68.4 ± 4.5 | 9.5 ± 1.2 | About 18 | About 200 |

It could be seen from the table that the BDDE-cross-linked silk hard bone material had a flexural strength of 68.4 MPa and an elastic modulus of 9.5 GPa, which was much lower than that of the stainless steel material (about 200 GPa) and closer to the elastic modulus of bone (about 18 GPa) than the metal material. Therefore, the BDDE-cross-linked silk hard bone material is more in line with the current concept of orthopaedic internal fixation.

Six healthy rabbits were selected and grouped, with 2 rabbits in each group. The rabbits were weighed and then anesthetized with 3% sodium pentobarbital by ear vein injection. Routine skin preparation and disinfection were carried out, the skin was incised at the rabbit's femoral ankles on both sides, and then the muscles were pulled apart to expose the femurs. Bone cortexes on the opposite sides were drilled through with an electric drill and then tapped, the BDDE-cross-linked silk hard bone materials were then screwed in. Subcutaneous suturing and disinfection were carried out. Gentamicin (10,000 units/kg) was administered by intramuscular injection 3 days after the operation. Two animals were sacrificed at first, second, and third months, and their femurs were taken out Animal sacrificing method: 20 mL of air was rapidly injected with a 20 mL syringe from the marginal ear vein of the rabbit to form an air embolism. In the test, a total of 12 hard bone materials were implanted into the femurs of the bilateral lower limbs of 6 rabbits, all of which were successfully implanted. The wounds healed well. All the rabbits had good mobility except one rabbit which was limp in the left hind leg. After the specimens were taken out at the first, second, and third months, it was found that the hard bone materials were still firmly fixed in the femoral shafts without prolapse or displacement. The contact surfaces between the materials and the bones were good, and there was no adverse reaction such as color change. This indicates that the BDDE-cross-linked silk hard bone materials have good machinability and can be successfully implanted into the femoral shafts of rabbits that have been drilled and tapped in advance. Within the first three months of implantation, it can be observed that the appearances of the specimens are relatively complete, and their hardness and toughness are still relatively high. It can be preliminarily determined that the fixation effect of the materials can last for more than 3 months.

The above-mentioned embodiments are only preferred embodiments for fully illustrating the present disclosure, and the scope of the present disclosure is not limited thereto. Equivalent substitutions or transformations made by those skilled in the art on the basis of the present disclosure are all within the scope of the present disclosure. The scope of the present disclosure is subject to the claims.

What is claimed is:

1. A chemically cross-linked hydrogel, the chemically cross-linked hydrogel being a hydrogel formed by reaction of silk with a crosslinking agent, wherein the crosslinking agent is a diglycidyl ether crosslinking agent, and a mass of silk fibroin accounts for 70% or above of a dry weight of a raw material of the silk.

2. The chemically cross-linked hydrogel according to claim 1, wherein the raw material of the silk is able to dissolve in an aqueous solution of lithium bromide.

3. The chemically cross-linked hydrogel according to claim 1, wherein the diglycidyl ether crosslinking agent is selected from the group consisting of diglycidyl ether, 1,4-butanediol diglycidyl ether, 1,3-diglycidyl glyceryl ether, bisphenol A diglycidyl ether and its derivatives, resorcinol diglycidyl ether, tris(4-hydroxyphenyl)methane triglycidyl ether, neopentyl glycol diglycidyl ether, and combinations thereof.

4. A preparation method of a chemically cross-linked hydrogel, comprising the following steps:
S1, dissolving silk fibers in an aqueous solution of lithium bromide to obtain a mixed solution containing silk; and
S2, adding a diglycidyl ether crosslinking agent to the mixed solution obtained in step S1 to carry out a crosslinking reaction to obtain the chemically cross-linked hydrogel.

5. The preparation method according to claim 4, wherein in step S1, a mass of silk fibroin in the silk fibers is 70% or above of the dry weight of the silk fibers; the silk fibers are able to dissolve in an aqueous solution of lithium bromide.

6. The preparation method according to claim 4, wherein in step S1, a concentration of lithium bromide in the mixed solution ranges from 1 M to 10 M.

7. The preparation method according to claim 4, wherein in step S1, a concentration of the silk in the mixed solution ranges from 1 mg/mL to 300 mg/mL.

8. The preparation method according to claim 4, wherein a ratio of the mass of the silk to the volume of the diglycidyl ether crosslinking agent is controlled to be 1 g:0.5 μL to 1 mL.

9. A type of chemically cross-linked hydrogel microspheres, the microspheres being hydrogel microspheres formed by reaction of silk with a crosslinking agent, wherein the crosslinking agent is a diglycidyl ether crosslinking agent.

10. The chemically cross-linked hydrogel microspheres according to claim 9, having a particle size ranging from 50 μm to 300 μm.

11. A preparation method of chemically cross-linked hydrogel microspheres, comprising the following steps:
S01, dissolving silk fibers in an aqueous solution of lithium bromide to obtain a mixed solution containing silk; and
S02, adding a diglycidyl ether crosslinking agent to the mixed solution obtained in step S01, and well mixing the resulting solution to obtain a reaction solution; and
S03, adding into an oil phase system the reaction solution obtained in step S02, and stirring the mixture to carry out a crosslinking reaction to obtain the chemically cross-linked hydrogel microspheres.

12. The preparation method according to claim 11, wherein in step S01, a concentration of lithium bromide in the mixed solution ranges from 1 M to 10 M.

13. The preparation method according to claim 11, wherein in step S01, a concentration of silk in the mixed solution ranges from 1 mg/mL to 300 mg/mL.

14. The preparation method according to claim 11, wherein in step S02, a ratio of the mass of the silk to the volume of the diglycidyl ether crosslinking agent is controlled to be 1 g:0.5 μL to 1 mL.

15. The preparation method according to claim 11, wherein in step S03, a volume ratio of the oil phase system to the reaction solution is greater than 1:1, and the stirring speed ranges from 100 rpm to 15000 rpm.

16. A hydrogel sphere composition, wherein a volume fraction of hydrogel particles and/or hydrogel microspheres in the composition ranges from 50% to 100%, and a mass fraction of silk ranges from 5% to 20%, wherein the hydrogel particles and/or the hydrogel microspheres are formed by reaction of silk with a crosslinking agent, and the crosslinking agent is a diglycidyl ether crosslinking agent.

17. The hydrogel sphere composition according to claim 16, further comprising one or more of a stabilizer, a lubricant and an osmotic pressure regulator.

18. The hydrogel sphere composition according to claim 16, further comprising one or more of a bioactive reagent, an extracellular matrix, a cell and a drug.

19. An application of the chemically cross-linked hydrogel according to claim 1 in tissue engineering filling, repair and/or drug delivery and in preparation of thin films, scaffolds or hard bone materials.

20. The application according to claim 19, further comprising arthritis treatment, medical cosmetic surgery or ophthalmic disease treatment.

21. An application of the chemically cross-linked hydrogel microspheres according to claim 9 in tissue engineering filling, repair and/or drug delivery and in preparation of thin films, scaffolds or hard bone materials.

22. An application of the gel sphere composition according to claim 16 in tissue engineering filling, repair and/or drug delivery and in preparation of thin films, scaffolds or hard bone materials.
